(19) United States
(12) Patent Application Publication
(10) Pub. No.: US 2005/0084490 A1
(43) Pub. Date: Apr. 21, 2005

(54) Boroproline compound combination therapy

(75) Inventors: Sharlene Adams, Waltham, MA (US); Glenn T. Miller, Merrimac, MA (US); Michael I. Jesson, Hopedale, MA (US); Barry Jones, Cambridge, MA (US)

Correspondence Address: Maria A. Trevisan;Wolf, Greenfield & Sacks, P.C., 600 Atlantic Avenue, Boston, MA, 02210, US

(73) Assignee: Point Therapeutics, Inc., Boston, MA (US)

(21) Appl. No.: 10/616,409

(22) Filed: Jul. 9, 2003

Related U.S. Application Data
(60) Provisional application No. 60/394,856, filed on Jul. 9, 2002.
(60) Provisional application No. 60/414,978, filed on Oct. 1, 2002.
(60) Provisional application No. 60/466,435, filed on Apr. 28, 2003.

(51) Int. Cl. A61K 39/395

(52) U.S. Cl. 424/144.1

(57) ABSTRACT

A method is provided for treating subjects with combination therapy including compounds of Formula I. It was surprisingly discovered that this combination enhanced the efficacy of both agents, and that administration of Formula I compounds induced cytokine and chemokine production in vivo. The combinations can be used to enhance ADCC, stimulate immune responses and/or patient and treat certain disorders. The invention also relates to kits and compositions relating to such combinations.

### RELATED APPLICATIONS

This application claims priority under 35 U.S.C. §119(e) to U.S. Provisional Application Ser. No. 60/394,856, entitled “BOROPROLINE COMPOUND COMBINATION THERAPY”, filed on Jul. 9, 2002; U.S. Provisional Application Ser. No. 60/414,978, entitled “BOROPROLINE COMPOUND COMBINATION THERAPY”, filed on Oct. 1, 2002; and U.S. Provisional Application Ser. No. 60/466,435, entitled “BOROPROLINE COMPOUND COMBINATION THERAPY”, filed on Apr. 28, 2003, each of which is herein incorporated by reference in its entirety.

### FIELD OF THE INVENTION

This invention relates to methods for the treatment and prevention of disorders by enhanced immunostimulation using DPIV inhibitors.

### BACKGROUND OF THE INVENTION

Cancer is the second leading cause of death, resulting in one out of every four deaths, in the United States. In 1997, the estimated total number of new diagnoses for lung, breast, prostate, colorectal and ovarian cancer was approximately two million. Due to the ever increasing aging population in the United States, it is reasonable to expect that rates of cancer incidence will continue to grow.

Cancer is currently treated using a variety of modalities including surgery, radiation therapy and chemotherapy. The choice of treatment modality will depend upon the type, location and dissemination of the cancer. One of the advantages of surgery and radiation therapy is the ability to control to some extent the impact of the therapy, and thus to limit the toxicity to normal tissues in the body. Chemotherapy is arguably the most appropriate treatment for disseminated cancers such as leukemia and lymphoma as well as metastases. Chemotherapy is generally administered systemically and thus toxicity to normal tissues is a major concern. Not all tumors, however, respond to chemotherapeutic agents and others, although initially responsive to chemotherapeutic agents, may develop resistance. As a result, the search for effective anti-cancer drugs has intensified in an effort to find even more effective agents with less non-specific toxicity.

Recently, much emphasis has been placed on the use of immunotherapy for the treatment and prevention of cancer and other disorders, including infectious disease. Immunotherapy provides the cell specificity that other treatment modalities lack. Methods for enhancing the efficacy of immune based therapies would be beneficial.

### SUMMARY OF THE INVENTION

The invention provides compositions and methods of use in the prevention and treatment of disorders that would benefit from enhanced immunostimulation. The invention is based, in part, on the surprising observation that the compounds of Formula I, either in linear or cyclic form, stimulate the production of cytokines and chemokines that can in turn stimulate immune cells. It has been found, according to the invention, that the compounds of Formula I stimulate the production of IL-1α, IL-1β, MCP-2, MARC/MCP-3, MCP-5, JBE, G-CSF, MIP-2, IL-8 (KC in mice), ENA78, LIX, lymphotactin, eotaxin, IL-6, MIG, IP-10, MDC, TARC, and thrombospondin, among others. Some of these cytokines activate macrophages and other antigen presenting cells, and thus are useful in enhancing immune responses that involve such cells including antibody dependent cell-mediated cytotoxicity and antigen presentation.

The ability of these compounds to stimulate cytokine and chemokine production endogenously is beneficial since exogenous administration of some of these factors, such as for example, IL-1, has been associated with toxicity. Production of IL-1 endogenously, and particularly in induction profiles that allow for induction in the spleen and lymph nodes with no detection in the serum indicates that the agents of Formula I can be used to induce cytokines in a controlled manner, and thereby overcome toxicity problems. Although not intending to be bound by any particular mechanism, it is further proposed that induction of these cytokines from cells in vivo also indicates that feedback loops normally operating in vivo may be operative and can control cytokine levels.

The invention is therefore also based in part on the observation that compounds of Formula I can be administered with disease specific antibodies in order to enhance the efficacy of such antibodies. Again, although not intending to be bound by any particular mechanism, it is proposed that the production of cytokines following administration of Formula I compounds leads to the stimulation of immune cells, thereby enhancing the response mediated by the exogenously administered antibody.

The invention relates to methods and compositions for enhancing immune therapies for a number of indications, both in a therapeutic and a prophylactic sense. Immune therapies include but are not limited to passive immune therapies such as immunoglobulin administration, and active immune therapies such as vaccination with antigens alone or antigens in the context of dendritic cells. The methods are intended to treat or prevent various indications that would benefit from an enhanced immune response.

In important aspects of the invention, the agents of Formula I are administered with an antibody or antibody fragment, with an antigen and optionally with an adjuvant, or as stand alone compositions. In some embodiments, the immune response that is stimulated is a cell-mediated immune response involving T cells, NK cells, macrophages, and the like. In other embodiments, the immune response that is stimulated is a humoral response involving B cells and antibody production. Both types of responses can co-exist in yet other embodiments. In still other embodiments, the immune response is an innate immune response, while in others it is an adaptive immune response.

The aspects of the invention commonly involve compounds (or agents, as used interchangeably herein) of Formula I:
PR
wherein P is a targeting group which binds to the reactive site of post proline-cleaving enzyme, and wherein R is a reactive group capable of reacting with a functional group in a post proline cleaving enzyme, preferably in the reactive site of the post proline cleaving enzyme. P may be a peptide or a peptidomimetic. The reactive compound may be selected from the group consisting of organo boronates, organo phosphonates, fluoroalkylketones, alphaketos, N-peptiolyl-O-acylhydroxylamines, azapeptides, azetidines, fluoroolefins dipeptide isoesteres, peptidyl (alpha-aminoalkyl) phosphonate esters, aminoacyl pyrrolidine-2-nitriles and 4-cyanothiazolidides. In some important embodiments, the compounds of the invention are boro-proline compounds.

One group of Formula I compounds useful in the invention can be further defined by Formula II: 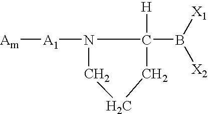
wherein m is an integer between 0 and 10, inclusive; A and A₁ may be L- or D-amino acid residues such that each A in Aₘ (i.e., where m>1) may be a different amino acid residue from every other A in Aₘ; the C bonded to B is in the L-configuration; the bond between A₁ and N and, in some embodiments, between A₁ and Aₘ, are peptide bonds; and each X₁ and X₂ is, independently, a hydroxyl group or a group capable of being hydrolyzed to a hydroxyl group in aqueous solution at physiological pH. By “the C bonded to B is in the L-configuration” is meant that the absolute configuration of the C is like that of an L-amino acid. Thus, the 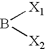
group has the same relationship to the C as the —COOH group of an L-amino acid has to its a carbon. In some embodiments, A and A₁ are independently proline or alanine residues. In some embodiments, m is 0. In some embodiments, X₁ and X₂ are hydroxyl groups.

In addition to agents of Formula II, other agents useful in the invention include those in which the proline residue in Formula II is replaced with another amino acid residue such as, for example, lysine, alanine or glycine. As well, derivatives of Formula II in which the boronate group is replaced with a reactive group as described above are also useful in the invention.

One group of Formula I compounds useful in the invention can be further defined by Formula III: 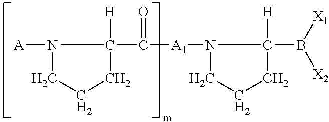
wherein m is an integer between 0 and 10, inclusive; A and A₁ are L- or D-amino acid residues; A in each repeating bracketed unit can be a different amino acid residue; the C bonded to B is in the L-configuration; the bonds between A and N, A₁ and C, and between A₁ and N are peptide bonds; and each X₁ and X₂ is, independently, a hydroxyl group or a group capable of being hydrolyzed to a hydroxyl group in aqueous solution at physiological pH.

In an important embodiment, the amino acids of these formulae are naturally occurring amino acids. Thus, in some embodiments, the agent is L-Ala-L-boroPro, L-Asp-L-boroPro, L-Glu-L-boroPro, L-Asn-L-boroPro, L-Gln-L-boroPro, L-Lys-L-boroPro, L-Arg-L-boroPro, L-His-L-boroPro, L-Pro-L-boroPro, L-Thr-L-boroPro, L-Ser-L-boroPro, L-Cys-L-boroPro, L-Gly-L-boroPro, L-Tyr-L-boroPro, L-Trp-L-boroPro, L-Phe-L-boroPro, L-Leu-L-boroPro, L-Ile-L-boroPro, L-Met-L-boroPro, or L-Val-L-boroPro. In some embodiments, the agent is L-Ile-L-boroPro, L-Met-L-boroPro, or L-Val-L-boroPro. In some preferred embodiments, the agent is L-Val-L-boroPro. In other embodiments, the amino acids of these formulae are non-naturally occurring or a mixture thereof.

Thus, in one aspect, the invention provides a method for stimulating an immune response in a subject comprising administering to a subject in need of immune stimulation an agent of Formula I, and an antibody or antibody fragment, in an amount effective to stimulate an immune response.

The aspects provided herein share a number of common embodiments. Accordingly, these embodiments will be recited once but it is to be understood that they apply equally to various related aspects of the invention.

In one embodiment, the agent of Formula I is an agent of Formula II. In another embodiment, the agent of Formula I is an agent of Formula III. In an important embodiment, the agent of Formula I is selected from the group consisting of L-Val-L-boroPro, L-Met-L-boroPro, and L-lie-L-boroPro. In another embodiment, the agent of Formula I is in a cyclic form. In yet another embodiment, the agent is optically pure.

Depending upon the aspect of the invention, the subject may be one in need of immune stimulation is a subject having or at risk of developing cancer. The cancer may be selected from the group consisting of a carcinoma and a sarcoma, but it is not so limited. In some important embodiments, the cancer is neither a carcinoma nor a sarcoma. In a related embodiment, the cancer is a leukemia or a lymphoma.

In one embodiment, the cancer is selected from the group consisting of basal cell carcinoma, biliary tract cancer; bladder cancer; bone cancer; brain cancer; breast cancer; cervical cancer; choriocarcinoma; CNS cancer; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; acute myeloid leukemia; acute lymphoid leukemia, chronic myeloid leukemia, chronic lymphoid leukemia, leukemia, liver cancer; small cell lung cancer; non-small cell lung cancer; lymphoma, Hodgkin's lymphoma; Non-Hodgkin's lymphoma; melanoma; myeloma; neuroblastoma; oral cavity cancer; ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; and cancer of the urinary system.

In another embodiment, the cancer is selected from the group consisting of bladder cancer, breast cancer, colon cancer, endometrial cancer, head and neck cancer, leukemia, lung cancer, lymphoma, melanoma, ovarian cancer, prostate cancer and rectal cancer.

In another embodiment, the cancer is a refractory cancer. Examples of refractory cancers include but are not limited to leukemias, melanomas, renal cell carcinomas, colon cancer, liver (hepatic) cancers, pancreatic cancer, Non-Hodgkin's lymphoma, and lung cancer. In still other embodiments, the cancer is an immunogenic cancer.

In still another embodiment, the cancer is a metastasis.

Depending upon the aspect of the invention, the subject is one in need of immune stimulation is a subject having or at risk of developing an infectious disease. The infectious disease may be selected from the group consisting of a bacterial infection, a inycobacterial infection, a viral infection, a fungal infection and a parasitic infection, but it is not so limited.

In one embodiment, the bacterial infection is selected from the group consisting of an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteriodes infection, H. pylori infection, and anthrax infection.

The mycobacterial infection may be tuberculosis or leprosy respectively caused by the M. tuberculosis and M. leprae species, but is not so limited.

In one embodiment, the viral infection is selected from the group consisting of an HIV infection, a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection and SARS infection.

In some important embodiments, the viral infection is not an HIV infection.

In yet another embodiment, the fungal infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

In another embodiment, the parasite infection is selected from the group consisting of amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginalis infection, Taenia infection, Hymenolepsis infection, Echinococcus infections, Schistosomiasis, neurocysticercosis, Necator americanus infection, and Trichuris trichuria infection.

In various aspects of the invention, the methods are intended to stimulate an immune response in a subject. In one embodiment, the immune response is antibody dependent cell-mediated cytoxicity. In another embodiment, the immune response is a cell-mediated immune response and/or a humoral (i.e., antibody-mediated) immune response. The immune response may be an innate immune response or an adaptive immune response, in other embodiments. In one embodiment, the immune response is an antigen specific immune response.

In some embodiment, the agent of Formula I is administered with or formulated with an antibody or antibody fragment. In one embodiment, the antibody or antibody fragment is an antibody.

The antibody or antibody fragment may be specific for a cell surface molecule. Cell surface molecules that may be targeted with the antibody or antibody fragment include but are not limited to HER 2, CD20, CD33, EGF receptor, HLA markers such as HLA-DR, CD52, CD1, CEA, CD22, GD2 ganglioside, FLK2/FLT3, VEGF, VEGFR, and the like.

The antibody or antibody fragment may be specific for a cancer antigen. Cancer antigens that may be targeted with the antibody or antibody fragment have been recited throughout the specification and include but are not limited to HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Other cancer antigens are described in U.S. Pat. No. 5,776,427. Still other cancer antigens are recited herein in Table 1.

Cancer antigens can be classified in a variety of ways. Cancer antigens include antigens encoded by genes that have undergone chromosomal alteration. Many of these antigens are found in lymphoma and leukemia. Even within this classification, antigens can be characterized as those that involve activation of quiescent genes. These include BCL-1 and IgH (Mantel cell lymphoma), BCL-2 and IgH (Follicular lymphoma), BCL-6 (Diffuse large B-cell lymphoma), TAL-1 and TCRδ or SIL (T-cell acute lymphoblastic leukemia), c-MYC and IgH or IgL (Burkitt lymphoma), MUN/IRF4 and IgH (Myeloma), PAX-5 (BSAP) (Immunocytoma).

Other cancer antigens that involve chromosomal alteration and thereby create a novel fusion gene and/or protein include RARoa, PML, PLZF, NPMor NuM4 (Acute promyelocytic leukemia), BCR and ABL (Chronic myeloid/acute lymphoblastic leukemia), MLL (HRX) (Acute leukemia), E2A and PBXor HLF (B-cell acute lymphoblastic leukemia), NPM, ALK (Anaplastic large cell leukemia), and NPM, MLF-1 (Myelodysplastic syndrome/acute myeloid leukemia).

Other cancer antigens are specific to a tissue or cell lineage. These include cell surface proteins such as CD20, CD22 (Non-Hodgkin's lymphoma, B-cell lymphoma, Chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (Epithelial and lymphoid malignancies), Human leukocyte antigen (HLA)-DR, HLA-DP, and HLA-DQ (Lymphoid malignancies), RCAS1 (Gynecological carcinomas, bilary adenocarcinomas and ductal adenocarcinomas of the pancreas), and Prostate specific membrane antigen (Prostate cancer).

Tissue- or lineage- specific cancer antigens also include epidermal growth factor receptors (high expression) such as EGFR (HER1 or erbB1) and EGFRvIII (Brain, lung, breast, prostate and stomach cancer), erbB2 (HER2 or HER2/neu) (Breast cancer and gastric cancer), erbB3 (HER3) (Adenocarcinoma), and erbB4 (HER4) (Breast cancer).

Tissue- or lineage- specific cancer antigens also include cell-associated proteins such as Tyrosinase, Melan-A/MART-1, tyrosinase related protein (TRP)-1/gp75 (Malignant melanoma), Polymorphic epithelial mucin (PEM) (Breast tumors), and Human epithelial mucin (MUC1) (Breast, ovarian, colon and lung cancers).

Tissue- or lineage- specific cancer antigens also include secreted proteins such as Monoclonal immunoglobulin (Multiple myeloma and plasmacytoma), Immunoglobulin light chains (Multiple Myeloma), α-fetoprotein (Liver carcinoma), Kallikreins 6 and 10 (Ovarian cancer), Gastrin-releasing peptide/bombesin (Lung carcinoma), and Prostate specific antigen (Prostate cancer).

Still other cancer antigens are cancer testis (CT) antigens that are expressed in some normal tissues such as testis and in some cases placenta. Their expression is common in tumors of diverse lineages and as a group the antigens form targets for immunotherapy. Examples of tumor expression of CT antigens include MAGE-A1, -A3, -A6, -A12, BAGE, GAGE, HAGE, LAGE-1, NY-ESO-1, RAGE, SSX-1, -2, -3, -4, -5, -6, -7, -8, -9, HOM-TES-14/SCP-1, HOM-TES-85 and PRAME. Still other examples of CT antigens and the cancers in which they are expressed include SSX-2, and -4 (Neuroblastoma), SSX-2 (HOM-MEL-40), MAGE, GAGE, BAGE and PRAME (Malignant melanoma), HOM-TES-14/SCP-1 (Meningioma), SSX-4 (Oligodendrioglioma), HOM-TES-14/SCP-1, MAGE-3 and SSX-4 (Astrocytoma), SSX member (Head and neck cancer, ovarian cancer, lymphoid tumors, colorectal cancer and breast cancer), RAGE-1, -2, -4, GAGE-1-2, -3, -4, -5, -6, -7 and -8 (Head and neck squamous cell carcinoma (HNSCC)), HOM-TES14/SCP-1, PRAME, SSX-1 and CT-7 (Non-Hodgkin's lymphoma), and PRAME (Acute lymphoblastic leukemia (ALL), acute myelogenous leukemia (AML) and chronic lymphocytic leukemia (CLL)).

Other cancer antigens are not specific to a particular tissue or cell lineage. These include members of the carcinoembryonic antigen (CEA) family: CD66a, CD66b, CD66c, CD66d and CD66e. These antigens can be expressed in many different malignant tumors and can be targeted by immunotherapy.

Still other cancer antigens are viral proteins and these include Human papilloma virus protein (cervical cancer), and EBV-encoded nuclear antigen (EBNA)-1 (lymphomas of the neck and oral cancer).

Still other cancer antigens are mutated or aberrantly expressed molecules such as but not limited to CDK4 and beta-catenin (melanoma).

The invention embraces the use of antibodies or antibodies fragments specific for any of the foregoing cancer antigens.

The antibody or antibody fragment may be specific for a stromal cell molecule. Stromal cell molecules that may be targeted with the antibody or antibody fragment include but are not limited to FAP and CD26.

The antibody or antibody fragment may be specific for an extracellular matrix molecule. Extracellular matrix molecules that may be targeted with the antibody or antibody fragment include but are not limited to collagen, glycosaminoglycans (GAGs), proteoglycans, elastin, fibronectin and laminin.

The antibody or antibody fragment may be specific for a tumor vasculature molecule. Tumor vasculature molecules include but are not limited to endoglin, ELAM-1, VCAM-1, ICAM-1, ligand reactive with LAM-1, MHC class II antigens, aminophospholipids such as phosphatidylserine and phosphatidylethanolamine, VEGFR1 (Flt-1) and VEGFR2 (KDR/Flk-1).

Antibodies to endoglin include TEC-4 and TEC-11. Antibodies that inhibit VEGF include 2C3 (ATCC PTA 1595). Other antibodies that are specific for tumor vasculature include antibodies that react to a complex of a growth factor and its receptor such as a complex of FGF and the FGFR or a complex of TGFβ and the TGFβR. Antibodies of this latter class include GV39 and GV97.

In a related embodiment, the antibody or antibody fragment is selected from the group consisting of trastuzumab, alemtuzumab (B cell chronic lymphocytic leukemia), gemtuzumab ozogamicin (CD33+ acute myeloid leukemia), hP67.6 (CD33+ acute myeloid leukemia), infliximab (inflammatory bowel disease and rheumatoid arthritis), etanercept (rheumatoid arthritis), rituximab, tositumomab, MDX-210, oregovomab, anti-EGF receptor mAb, MDX-447, anti-tissue factor protein (TF), (Sunol); ior-c5, c5, edrecolomab, ibritumomab tiuxetan, anti-idiotypic mAb mimic of ganglioside GD3 epitope, anti-HLA-Dr10 mAb, anti-CD33 humanized mAb, anti-CD52 humAb, anti-CD1 mAb (ior t6), MDX-22, celogovab, anti-17-1A mAb, bevacizumab, daclizumab, anti-TAG-72 (MDX-220), anti-idiotypic mAb mimic of high molecular weight proteoglycan (I-Mel-1), anti-idiotypic mAb mimic of high molecular weight proteoglycan (I-Mel-2), anti-CEA Ab, hmAbH11, anti-DNA or DNA-associated proteins (histones) mAb, Gliomab-H mAb, GNI-250 mAb, anti-CD22, CMA 676), anti-idiotypic human mAb to GD2 ganglioside, ior egf/r3, anti-ior c2 glycoprotein mAb, ior c5, anti-FLK-2/FLT-3 mAb, anti-GD-2 bispecific mAb, antinuclear autoantibodies, anti-HLA-DR Ab, anti-CEA mAb, palivizumab, bevacizumab, alemtuzumab, BLyS-mAb, anti-VEGF2, anti-Trail receptor; B3 mAb, mAb BR96, breast cancer; and Abx-Cbl mAb.

In one important embodiment, the antibody or antibody fragment is an anti-HER2 antibody, and preferably it is trastuzumab. In another important embodiment, the antibody or antibody fragment is an anti-CD20 antibody, and preferably it is rituximab.

The antibody or antibody fragment may conjugated (covalently or otherwise) to a toxin derived from plant, fungus, or bacteria. The toxin may be selected from the group consisting of A chain toxin, deglycosylated A chain toxin, ribosome inactivating protein, α-sarcin, aspergillin, restrictocin, ribonuclease, diptheria toxin and Pseudomonas exotoxin, but is not so limited.

The antibody or antibody fragment may also conjugated to a chemotherapeutic agent, a radioisotope or a cytotoxin. The chemotherapeutic agent may be selected from the group consisting of an anti-metabolite, an anthracycline, a vinca alkaloid, an antibiotic, an alkylating agent, and an epipodophyllotoxin, but is not so limited.

In one embodiment, the antibody or antibody fragment is administered in a sub-therapeutic dose.

In various embodiments, the agent of Formula I is administered on a routine schedule.

In one embodiment, the agent of Formula I is administered in a route of administration different from that of the antibody or antibody fragment.

In still other embodiments, the subject is otherwise free of symptoms calling for hematopoietic stimulation. The subject may be non-immunocompromised, but is not so limited. In some embodiments, the subject is genetically immunocompromised, and may be so as a result of a genetic mutation such as in agammaglobulinemia or SCID. In another embodiment, the subject may have an immune deficiency selected from the group consisting of Bruton's agammaglobulinemia, congenital hypogammaglobulinemia, common variable immunodeficiency, and selective immunoglobulin A deficiency. In another embodiment, the subject is elderly (e.g., at least 50 years old). In still another embodiment, the subject is non-immunocompromised as it has not undergone any immunosuppressive therapies such as chemotherapy or radiation.

In one embodiment, the agent of Formula I is administered orally and the antibody or antibody fragment is administered by injection. In another embodiment, the agent of Formula I is administered prior to the antibody or antibody fragment. In still another embodiment, the agent of Formula I is administered in an amount that increases lymphoid tissue (e.g., spleen) levels of IL-1, G-CSF or IL-8 (KC in mice). In the various embodiments described herein, it is to be understood that the invention embraces induction of either or both IL-1α and IL-1β, and thus a general recitation of IL-1 means both α and β forms. In another embodiment, the agent of Formula I is administered in an amount that does not increase serum IL-1 levels.

In one embodiment, the agent of Formula I is administered 30 minutes to 8 hours prior to the antibody or antibody fragment. In another embodiment, the agent of Formula I is administered 1 to 7 days prior to the antibody or antibody fragment. In yet another embodiment, the agent of Formula I is administered substantially simultaneously with the antibody or antibody fragment. As used herein, the term “substantially simultaneously” means that the compounds are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably.

In one embodiment, the agent of Formula I is administered after the antibody or antibody fragment.

The antibody or antibody fragment may be administered on a first day of multi-day cycle, with the agent of Formula I administered on the remaining days of the cycle. The cycle may be a 2, 3, 4, 5, 6, 7, or more day cycle. The agent of Formula I may be administered once, twice, or more times per day. In one embodiment, the antibody or antibody fragment is administered on the first day of a seven day cycle, followed by a twice daily administration of the agent of Formula I on each of the remaining days of the seven day cycle.

The multi-day cycle may be repeated twice, thrice, four times, or more. It may also be repeated for various lengths of time, including but not limited to a week, a month, two months, or more.

The compositions of the invention may be provided in a housing such as a container, a box, or a bag. The housing may also contain instructions for use of the composition either thereon or therein. The instructions for use indicate how the contents of the housing are to be used, including timing and dose of administrations. In these latter embodiments, the compositions may be contained in a kit.

In another aspect, the invention provides a method for stimulating an immune response in a subject comprising administering to a subject in need of immune stimulation an agent of Formula I, and an antigen, in an amount effective to stimulate an antigen-specific immune response, wherein the agent of Formula I is administered at a concentration of greater than 10⁻⁸M.

In one embodiment, the subject is HIV negative.

In one embodiment, the agent of Formula I is administered on a routine schedule. In another embodiment, the agent of Formula I is administered in a route of administration different from that of the antigen.

In another embodiment, the method further comprises administering an adjuvant to the subject. In one embodiment, the adjuvant is selected from the group consisting of alum, cholera toxin, CpG immunostimulatory nucleic acids, MPL, MPD, and QS-2 1.

In one embodiment, the antigen is a cancer antigen. The cancer antigen may be selected from the group consisting of MART-1/Melan-A, gp100, adenosine deaminase-binding protein (ADAbp), FAP, cyclophilin b, colorectal associated antigen (CRC)-C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, and CD20. The cancer antigen may also be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). In still another embodiment, the cancer antigen is selected from the group consisting of GAGE-1, GAGE-2, GAGE-3, GAGE-4, GAGE-5, GAGE-6, GAGE-7, GAGE-8, GAGE-9. And in yet a further embodiment, the cancer antigen is selected from the group consisting of BAGE, RAGE, LAGE-1, NAG, GnT-V, MUM-1, CDK4, tyrosinase, p53, MUC family, HER2/neu, p21ras, RCAS 1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-l and CT-7, and c-erbB-2.

The cancer also includes any of the cancer antigens mentioned infra with respect to other aspects of the invention, such as for example those listed in Table 1.

In certain embodiments of the foregoing aspects of the invention, the methods may further comprise treating the subject with a therapy selected from the group consisting of surgery, radiation and chemotherapy.

In one embodiment, the agent of Formula I and the antigen (or the antibody) are administered prior to treating the subject with a therapy selected from the group consisting of surgery, radiation and chemotherapy. In another embodiment, the agent of Formula I and the antigen (or antibody) are administered after treating the subject with a therapy selected from the group consisting of surgery, radiation and chemotherapy. In yet another embodiment, the agent of Formula I and the antigen (or antibody) are administered before and after treating the subject with a therapy selected from the group consisting of surgery, radiation and chemotherapy.

In one embodiment, the agent of Formula I is administered to the subject prior to the antigen (or the antibody). In another embodiment, the agent of Formula I is administered to the subject 30 minutes to 8 hours before administration of the antigen (or the antibody). In another embodiment, the agent of Formula I is administered to the subject 1 to 7 days before administration of the antigen (or the antibody).

In another embodiment, the agent of Formula I is administered to the subject after the antigen (or the antibody). In another embodiment, the agent of Formula I is administered to the subject 30 minutes to 8 hours after administration of the antigen (or the antibody). In another embodiment, the agent of Formula I is administered to the subject 1 to 7 days after administration of the antigen (or the antibody).

In one embodiment, the antigen is a microbial antigen. As used herein, a microbial antigen is an antigen derived from an infectious pathogen, and may include the entire pathogen. The antigen may be peptide, lipid, or carbohydrate in nature, but it is not so limited.

In one embodiment, the microbial antigen is selected from the group consisting of a bacterial antigen, a mycobacterial antigen, a viral antigen, a fungal antigen, and a parasitic antigen.

In one embodiment, the bacterial antigen is derived from a bacterial species selected from the group consisting of E. coli, Staphylococcal, Streptococcal, Pseudomonas, Clostridium difficile, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and anthrax.

The mycobacterial antigen may be derived from a mycobacterial species such as M. tuberculosis and M. leprae, but is not so limited.

In another embodiment, the viral antigen is derived from a viral species selected from the group consisting of HIV, Herpes simplex virus 1, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS.

In yet another embodiment, the fungal antigen is derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

In still another embodiment, the parasitic antigen is derived from a parasite species selected from the group consisting of amebiasis, Trypanosoma cruzi, Fascioliasis, Leishmaniasis, Plasmodium, Onchocerciasis, Paragonimiasis, Trypanosoma brucei, Pneumocystis, Trichomonas vaginalis, Taenia, Hymenolepsis, Echinococcus, Schistosomiasis, neurocysticercosis, Necator americanus, and Trichuris trichuria.

The invention intends to embrace various antigens from the infectious pathogens recited herein.

The invention provides in yet another aspect a composition comprising an effective amount of an agent of Formula I and an antibody or antibody fragment. In one embodiment, the composition further comprises a pharmaceutically acceptable carrier.

In one embodiment, the effective amount is an amount to stimulate antibody dependent cell-mediated cytoxicity. In another embodiment, the effective amount is an amount to treat or prevent cancer. In still another embodiment, the effective amount is an amount to treat or prevent an infectious disease.

In one embodiment, the antibody or antibody fragment is an antibody, and it can be selected from the group listed above.

In another aspect, the invention provides a composition comprising an effective amount of an agent of Formula I and a cancer antigen. In one embodiment, the effective amount is an amount to treat or prevent cancer.

In this and other aspects of the invention, the cancer antigen may be a peptide antigen, or a lipid antigen, but it is not so limited. The cancer antigen can be selected from the groups recited above. In one embodiment, the agent of Formula I is formulated for administration at a dose of greater than 10⁻⁸M.

In yet another aspect, the invention provides a method of preventing an infectious disease in a subject at risk of developing an infectious disease comprising identifying a subject at risk of developing an infectious disease, and administering an agent of Formula 1 to the subject in an amount effective to induce IL-1.

In one embodiment, the method further comprises administering to the subject a microbial antigen, selected from the groups recited above. In one embodiment, the infectious disease is selected from the group consisting of a bacterial infection, a viral infection, a fungal infection and a parasitic infection, and these can be selected from the groups listed above.

In one embodiment, the subject is HIV negative. In one embodiment, the viral infection is selected from the group consisting of a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection and SARS infection.

In yet another aspect, the invention provides a composition comprising an effective amount of an agent of Formula I and a microbial antigen, wherein the agent of Formula I is formulated for administration at a dose of greater than 10⁻⁸M. In one embodiment, the effective amount is an amount to treat or prevent an infectious disease.

The microbial antigen can be selected from the groups recited above.

In yet another aspect, the invention provides a method for stimulating an immune response in a subject having or at risk of having cancer comprising administering to a subject in need of immune stimulation an agent of Formula I, and an antigen, in an amount effective to stimulate an antigen-specific immune response.

In one embodiment, the subject is HIV negative. In another embodiment, the subject is a subject having cancer. In yet another embodiment, the cancer is selected from the group consisting of a lymphoma or leukemia. In still other embodiments, the cancer may be selected from the groups recited above. In one embodiment, the cancer is a metastasis. In yet another embodiment, the subject has or is at risk of developing an infectious disease, and these infectious diseases can be selected from the groups recited above. In one embodiment, the subject is further administered an antigen such as a cancer antigen or a microbial antigen, and either can be selected from the groups recited above.

In one embodiment, the method further comprises treating the subject with a therapy selected from the group consisting of surgery, radiation and chemotherapy.

In one embodiment, the agent of Formula I and the antigen are administered prior to treating the subject with a therapy selected from the group consisting of surgery, radiation and chemotherapy. In another embodiment, the agent of Formula I and the antigen are administered after treating the subject with a therapy selected from the group consisting of surgery, radiation and chemotherapy. In another embodiment, the agent of Formula I and the antigen are administered before and after treating the subject with a therapy selected from the group consisting of surgery, radiation and chemotherapy.

In still another embodiment, the agent of Formula I is administered to the subject prior to the antigen. In a related embodiment, the agent of Formula I is administered to the subject 30 minutes to 8 hours before administration of the antigen. In still another embodiment, the agent of Formula I is administered to the subject 1 to 7 days before administration of the antigen.

In one embodiment, the agent of Formula I is administered in an amount that increases lymphoid tissue (e.g., spleen) levels of IL-1, G-CSF or IL-8 (KC in mice). In another embodiment, the agent of Formula I is administered in an amount that does not increase serum IL-1 levels in one embodiment, the agent of Formula I is administered in a dose of greater than 10⁻⁸M.

In one embodiment, the subject is further administered an adjuvant, and the adjuvant is optionally selected from the group consisting of alum, cholera toxin, CpG immunostimulatory nucleic acids, MPL, MPD, and QS-21.

In yet another embodiment, the subject has not undergone an anti-cancer therapy selected from the group consisting of surgery, radiation and chemotherapy.

The invention provides in still another aspect, a method for stimulating an immune response in a non-immunocompromised subject comprising administering to a subject in need thereof an agent of Formula I, in an amount effective to induce IL-1. The IL-1 can be IL-1α or IL-1β.

In one embodiment, the method can further comprise administering an antigen or an antibody or fragment thereof to the subject. The antigen can be a cancer antigen or a microbial antigen, as taught herein, but it is not so limited.

In one embodiment, the subject will have a surgery. In another embodiment, the subject has a skin abrasion from a trauma. In yet another embodiment, the subject is traveling to a region in which a microbial infection is common. In one embodiment, the subject is elderly.

In one embodiment, the agent of Formula I and the antigen are formulated together.

In another embodiment, the antigen is administered mucosally. In one embodiment, the agent of Formula I is administered orally. In another embodiment, the antigen and the agent of Formula I are both administered mucosally.

In still another aspect of the invention, a method is provided for stimulating an immune response in a genetically immunocompromised subject comprising administering to a subject in need thereof an agent of Formula I, in an amount effective to induce IL-1.

In one embodiment, the subject has a genetic deficiency selected from the group consisting of SCID, agammaglobulinemia such as Bruton's agammaglobulinemia and congenital hypogammaglobulinemia, common variable immunodeficiency (CDG), and selective immunoglobulin A deficiency.

In yet a further aspect of the invention, a method is provided for treating a subject having or at risk of developing an interferon (IFN)-responsive condition. The method comprises administering to a subject in need of such treatment an agent of Formula I in

an amount effective to induce a therapeutically or prophylactically effective amount of IL-1 in the subject. The method may further comprise identification of a subject having or at risk of developing an IFN-responsive condition. The IFN may be IFNα, IFNα-2b, IFNβ or IFNγ, but is not so limited. In one embodiment, the condition is an IFNγ-responsive condition, and may be selected from the group consisting of viral infections and associated diseases, and cancer. In one embodiment, the subject is HIV positive. In one embodiment, the IFN-responsive condition is a chronic infection selected from the group consisting of a chronic hepatitis B infection, chronic hepatitis C infection, chronic Epstein Barr Virus infection, and tuberculosis. Other disorders include hepatocellular carcinoma, Kaposi's Sarcoma (AIDS-related), thick primary melanomas, and regional lymph node metastases. In one embodiment, the disorder is refractive (i.e., resistant) to prior therapy (e.g., drug treatment) Thus, in one embodiment, the disorder is drug resistant. In another embodiment, the disorder is multiple sclerosis. IFN-responsive conditions are not intended to be so restricted however.

In one embodiment, the IL-1 is IL-1α or IL-1β. In another embodiment, the method further comprises administering to the subject a second active agent selected from the group consisting of IFNα, pegylated IFN, IFNα-2b, acyclovir, lobucavir, ganciclovir, L-deoxythymidine, clevudine, a therapeutic vaccine, phosphonoformate (PFA), ribavirin (RBV), thymosin alpha-1, 2 3-dideoxy-3-fluoroguanosine (FLG), famciclovir, lamivudine, adefovir dipivoxiI, entecavir, emtricitabine, and hepatitis B-specific immunoglobulin.

In a further aspect, the invention provides a method for treating a subject having or at risk of developing cancer comprising administering to a subject in need of such treatment an enzyme inhibitor selected from the group consisting of a tyrosine kinase inhibitor, a CDK inhibitor, a MAP kinase inhibitor, and an EGFR inhibitor, and an agent of Formula I in an amount effective to inhibit the cancer. In one embodiment, the tyrosine kinase inhibitor is selected from the group consisting of Genistein (4′,5,7-trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-]-(3′-carboxy-4′-hydroxyphenyl)-2-(2″,5″-dihydroxy-phenyl)ethane, and HDBA (2-Hydroxy5-(2,5-Dihydroxybenzylamino)-2-hydroxybenzoic acid. In another embodiment, the CDK inhibitor is selected from the group consisting of p21, p27, p57, p15, p16, p18, and p19. In another embodiment the MAP kinase inhibitor is selected from the group consisting of KY 12420 (C₂₃H₂₄O₈), CNI-1493, PD98059, 4-(4-Fluorophenyl)-2-(4-methylsulfinyl phenyl)-5-(4-pyridyl) 1H-imidazole. In still a further embodiment, the EGFR inhibitor is selected from the group consisting of Tarceva™(OSI-774), Iressa (ZD1839), WHI-P97 (quinazoline derivative), LFM-A12 (leflunoinide metabolite analog), AG1458. In various embodiments, the amount effective is a synergistic amount.

In yet one more aspect of the invention, a method is provided for treating a subject having or at risk of developing cardiovascular disease comprising administering to a subject in need of such treatment an agent of Formula I in an amount effective to induce an effective amount of IL-1. The method may further comprise identifying a subject in need of such treatment.

In another aspect, the invention provides a method for preventing drug resistance in a subject. The method involves administering to a subject receiving an anti-microbial agent, an agent of Formula I in an amount effective to reduce the risk of resistance to the anti-microbial agent. In one embodiment, the subject is one having or is at risk of developing an infectious disease. As used herein, the terms “infectious disease” and “microbial infection” are used interchangeably and intended to convey an infection by any microbe including but not limited to a bacterium, a mycobacterium, a virus, a fungus, a parasite, and the like. Thus, in one embodiment, the infectious disease is selected from the group consisting of a bacterial infection, a viral infection, a fungal infection and a parasitic infection. In one embodiment, the bacterial infection is a Pseudomonas infection. Other drug resistant microbes and the drugs to which they are resistant include Staphylococcus aureus (penicillin), Streptococcus pneumoniae (penicillin), gonorrhea (penicillin), and Enterococcus faecium (penicillin). In one embodiment, the anti-microbial agent is selected from the group consisting of an anti-bacterial agent, an anti-viral agent, an anti-fungal agent, and an anti-parasitic agent.

In still another aspect, the invention provides a method for shortening a vaccination course. As used herein, “shortening a vaccination course” refers to reducing either the number of vaccine administrations (e.g., by injection) or the time between vaccine administrations. This is accomplished by stimulating a more robust immune response in the subject. The method may involve, in one embodiment, administering to a subject in need of immunization an agent of Formula I in an amount effective to induce an antigen-specific immune response to a vaccine administered in a vaccination course, wherein the vaccination course is shortened by at least one immunization. In other embodiments, the vaccination course is shortened by one immunization, two immunizations, three immunizations, or more. The method may involve, in another embodiment, administering to a subject in need of immunization an agent of Formula I in an amount effective to induce an antigen-specific immune response to a vaccine administered in a vaccination course, wherein the vaccination course is shortened by at least one day. In other embodiments, the vaccination course is shortened by one day, two days, three days, four days, five days, six days, one week, two weeks, three weeks, four weeks, one month, two months or more. In one embodiment, the agent of Formula I is administered substantially simultaneously with the vaccine. Immunizations that can be modified in this way include but are not limited to newborn immunizations for HBV; immunizations at for example two months of age for Polio, DTaP, Hib, HBV, Pneumococcus; immunizations at for example four months of age for Polio, DTaP, Hib, Pneumococcus; immunizations at for example six months of age for Polio, DTaP, Hib, HBV, Pneumococcus; immunizations at for example 12-15 months of age for Hib, Pneumococcus, MMR, Varicella; immunizations at for example 15-18 months of age for DtaP; immunizations at for example 4-6 years of age for Polio, DPT, MMR; immunizations at for example 11-12 years of age for MMR; immunizations at for example 14-16 years of age for tetanus-diphtheria (i.e., Td) (with a repeat as a booster every 10 years). As an example, a recommended vaccination course for tetanus/diphtheria includes a primary immunization series given in adults if not received as a child, followed by routine booster doses of tetanus-diphtheria (Td) every 10 years. The method of the invention will allow for a shortened series of vaccinations at the first time point, and may in some instances obviate the need for booster shoots later on. As another example, hepatitis vaccination commonly requires three administrations spaced at least two weeks, and sometimes one month, apart in order to develop full immunity. Using the methods of the invention, it is possible to either reduce the number of injections from three to two or one, or to reduce the time in between injections from weeks or months to days or weeks. Vaccination courses that can be shortened by the method of the invention include but are not limited to: HBV: Hepatitis B vaccine (3 total doses currently recommended); Polio: Inactivated polio vaccine (4 total doses currently recommended); DTaP: Diphtheria/tetanus/acellular Pertussis (3-in-1 vaccine; 5 total doses currently recommended); Hib: Haemophilus influenzae type b conjugate vaccine (4 total doses currently recommended); Pneumococcus (Prevnar): Protects against certain forms of Strep. Pneumoniae (3 total doses recommended); MMR: measles/mumps/rubella (3-in-1 vaccine; 2 total doses recommended); Td: Adult tetanus/diphtheria (2-in-i vaccine; for use in people over age 7). In another embodiment, the compounds of Formula I can be used together with oral polio vaccine.

The invention provides in yet another aspect a method for stimulating an immune response in a subject having cancer comprising administering to a subject in need of such treatment an agent of Formula I in an amount effective to stimulate an antigen-specific immune response, prior to and following a therapy selected from the group consisting of radiation, surgery and chemotherapy. The foregoing embodiments relating to agent of Formula I are equally applicable to this aspect of the invention. The foregoing embodiments relating to cancer are similarly equally applicable to this aspect of the invention.

In one embodiment, the subject is otherwise free of symptoms calling for hemopoietic stimulation. In one embodiment, the method further comprises administering an adjuvant to the subject. In another embodiment, the adjuvant is selected from the group consisting of alum, cholera toxin, CpG immunostimulatory nucleic acids, MPL, MPD, and QS-21.

In one embodiment, the agent of Formula I is administered to the subject 30 minutes to 8 hours before the therapy and 30 minutes to 8 hours after the therapy.

In another embodiment, the agent of Formula I is administered in an amount that increases lymphoid tissue (e.g., spleen) levels of IL-1, G-CSF or IL-8 (KC in mice). In another embodiment, the agent of Formula I is administered in an amount that does not increase serum IL-1 levels.

In one embodiment, the agent of Formula I is administered in a dose of greater than 10⁻⁸M.

In still another aspect, a method is provided for stimulating an immune response in a subject at risk of developing cancer comprising administering to a subject in need of such treatment an agent of Formula I in an amount effective to stimulate an antigen-specific immune response. In one embodiment, the method further comprises identifying a subject in need of such treatment. In another embodiment, the subject at risk of developing cancer has a familial predisposition to developing cancer. In one embodiment, the familial predisposition is familial colon polyposis. In a related embodiment, the subject has precancerous polyps. In another embodiment, the subject has precancerous HPV lesions. In other embodiments the familial predisposition can include BRCA1- and BRCA2-associated breast cancer, Wilms tumor, colorectal cancer, Li-Fraumeni Syndrome, ovarian cancer, and prostate cancer. In another embodiment, the subject is at risk of developing a cancer that is a metastasis.

These and other aspects of the invention will be described in greater detail below. Throughout this disclosure, all technical and scientific terms have the same meaning as commonly understood by one of ordinary skill in the art to which this invention pertains unless defined otherwise.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 is a histogram of cytokine and chemokine gene expression in normal lymph node and WEHI 164 tumor samples following exposure to PT-100 (i.e., Val-boroPro).

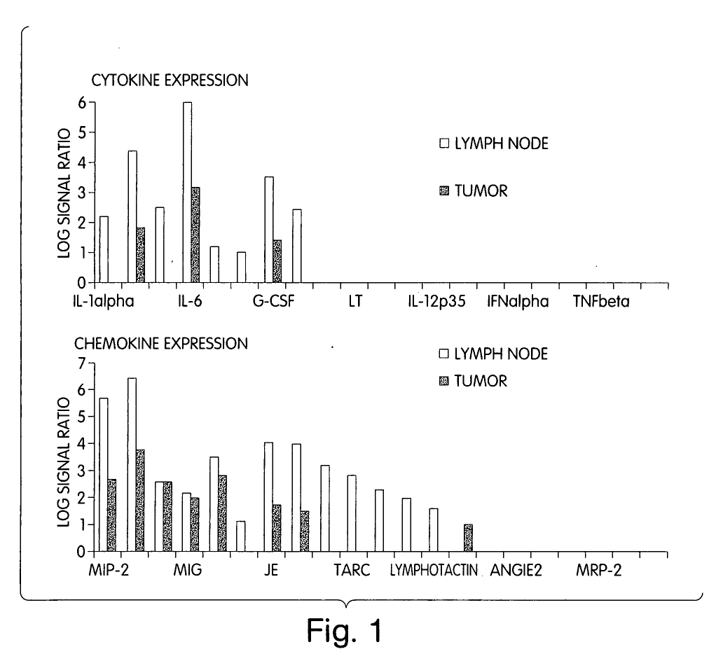

FIG. 2 is a graph of the effect of PT-100 inoculation (5 μg) on tumor volume as a function of time after inoculation in BALB/c +/+ (left panel) and BALB/c nu/nu mice (right panel).

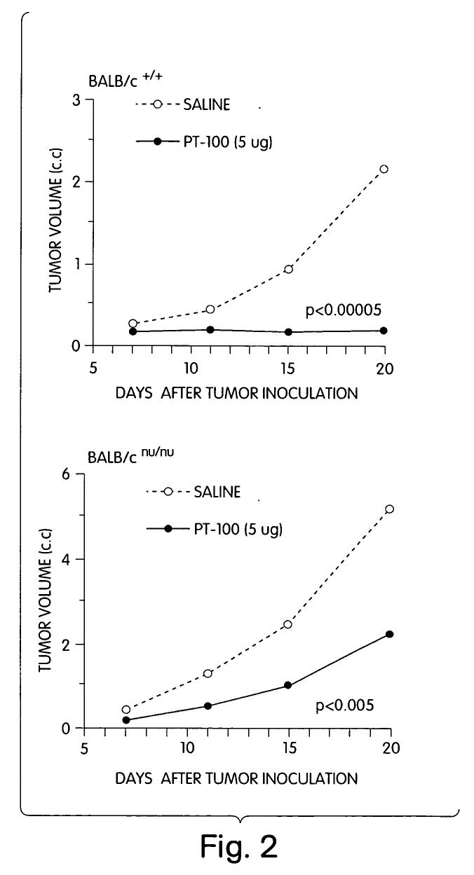

FIG. 3 is a graph of the effect of control IgG, PT-100 and control IgG, anti-CD20 antibody rituximab (Rituxan™) alone, and PT-100 and anti-CD20 antibody rituximab (Rituxan™) together on tumor volume in a NOD/SCID mouse model of Burkitt's Non-Hodgkin's Lymphoma as function of time after inoculation.

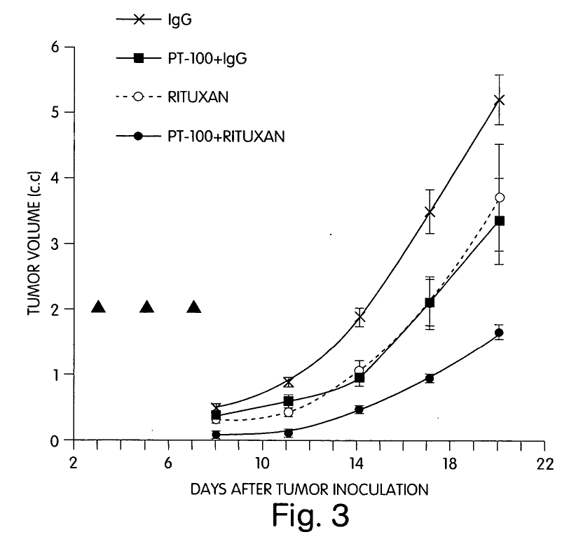

FIG. 4 is a histogram of cytokine induction at 30 minutes and 2 hours following administration of PT-100 in mice.

FIG. 5 is a set of histograms showing the induction of IL-1β, G-CSF and KC in serum and spleen following PT-100 administration (40 μg or 160 μg) in wild type mice and IL-1 receptor-1 mutant (−/−) mice.

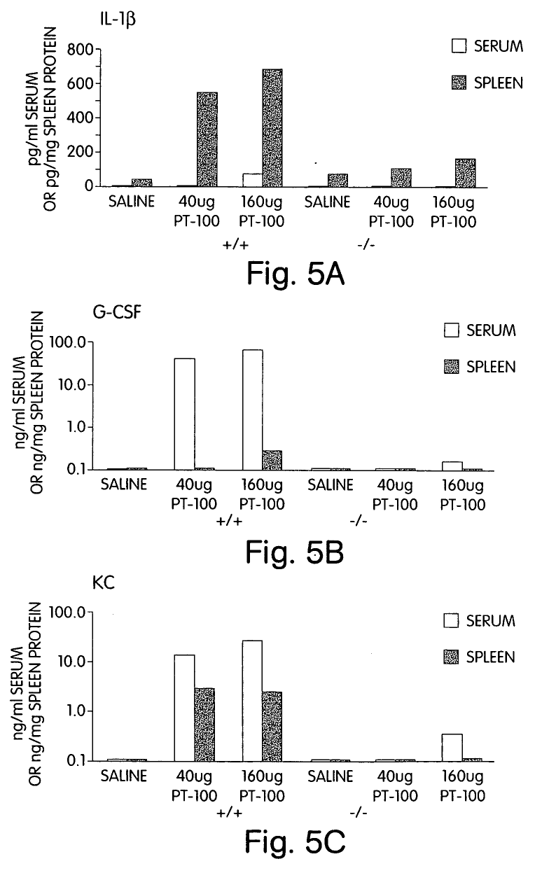

FIG. 6 is a set of histograms showing the induction of IL-1β, G-CSF and KC in serum and spleen of mice administered 20 μg PT-100.

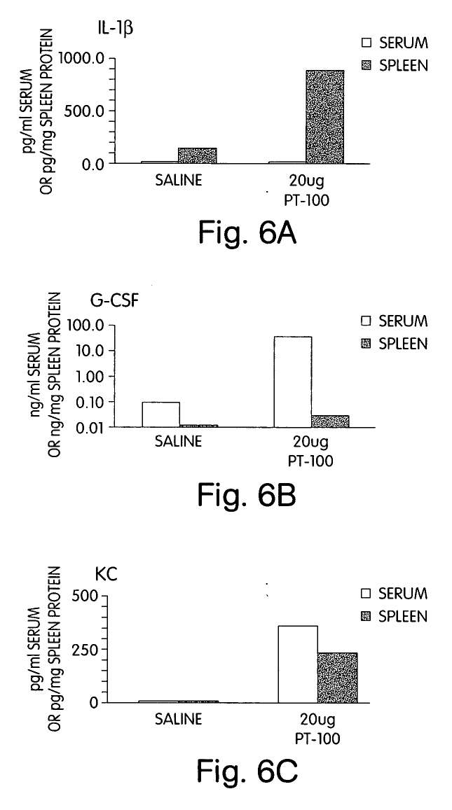

It is to be understood that the figures are not required for enablement of the invention.

### DETAILED DESCRIPTION OF THE INVENTION

The invention is based in part on the discovery that the agents of Formula I stimulate a variety of cytokines and chemokines which can stimulate the immune system. The resultant immune stimulation can thus be exploited to enhance the efficacy of immune based therapies such as passive (i.e., immunoglobulin) immunotherapy, or active immunization with antigens. Thus, in one aspect, the invention provides methods that exploit the synergy that is achieved when the compounds of Formula I are used together with antibodies or fragments thereof. In another aspect, the invention provides methods for stimulating an antigen specific immune response by administering the compounds of Formula I together with antigens. The antigens may be targeted to particular cell types or tissues (see, for example, Corixa targeted antigens). These antibodies and antigens that can be used in the methods of the invention are not restricted to those that are cancer specific, and as described in greater detail herein can apply to a broad range of conditions.

Thus in one aspect, the invention provides, in part, methods and products for the more effective treatment of cancer using agents of Formula I in combination with cancer specific antibodies. In one embodiment, the combination is synergistic, resulting in greater than additive effects than would otherwise be expected using the agents separately. In other embodiments, the combination is additive.

Antibodies specific for tumor or cancer antigens can suppress tumor growth in vivo via a variety of mechanisms. Antibody dependent cell-mediated cytotoxicity, complement mediated cell lysis, targeting of chemically linked toxins, inhibition of tumor cell division, and induction of tumor cell apoptosis have all been described as mechanisms by which immunoglobulins specific for tumor antigens suppress tumor growth in the treatment of cancer. Although antibody-based treatments for cancer can be effective, they do not completely suppress tumor development and progression in all subjects.

Compounds of Formula I can suppress a number of different mouse tumors in mice. It has now been demonstrated that these compounds, when administered to tumor-bearing mice, rapidly stimulate the production of growth factors, cytokines and chemokines. These mediators collectively stimulate the proliferation, activation and chemoattraction to the tumor microenvironment of effector cells involved in both non-adaptive (innate) and immune lysis or growth inhibition of tumor cells. The immune and non-immune effector cell populations mobilized and/or activated by compounds of Formula I enhance the tumor suppressive effects of anti-cancer antibodies.

Examples of effector cells involved in the anti-tumor effects of Formula I compounds are given below. Although not intending to be bound by any particular mechanism, a brief description of how each cell type can cooperate with tumor-specific antibodies in the lysis or growth inhibition of tumor cells is provided herein.

Tumor-infiltrating T cells, including cytotoxic T lymphocytes (CTL), that either lyse or inhibit tumor growth will suppress tumors by a mechanism of antigen-recognition that is different from that of antibodies. Thus, tumor-specific T cells can augment tumor cell lysis or growth inhibition initiated by antibody-based therapeutics.

Macrophage/monocyte, neutrophil, eosinophil, natural killer cells, and lymphokine activated killer cells are also activated by Formula I compounds. Individually or collectively, these effector cell types can either lyse tumor cells or suppress their growth in ligand-receptor mediated interactions that lack immunological specificity. The activities of these cells can account for the innate or non-adaptive immune responses against tumors stimulated by Formula I compounds. In addition, all of these cell types possess receptors that bind to the Fc portion of immunoglobulin and are referred to as Fc receptors. Fc receptors can bind to antibodies that are specifically bound to tumor cells by their antigen-binding regions. Therefore, since each effector cell possesses cytotoxicity or growth inhibitory activity against tumor cells, the antibody-mediated interaction targets this activity specifically against the tumor. The mechanism can therefore increase the efficiency with which these otherwise non-specific effector cells suppress tumor growth. The process is frequently referred to as antibody dependent cell-mediated cytotoxicity (ADCC).

Thus, in one aspect, the invention provides a method for stimulating ADCC in a subject. The method comprises administering an anti-cancer antibody or antibody fragment and a compound of Formula I to a subject having or at risk of developing cancer in an amount effective to stimulate antibody dependent cell-mediated cytotoxicity in the subject. In some embodiments, the amount effective to stimulate antibody dependent cell-mediated cytotoxicity is a synergistic amount.

In another aspect, the invention provides methods for inducing mucosal immunity. The mucosal surface is frequently in contact with infectious pathogens such as bacteria, viruses and fungi, and thus an enhanced immune response at this surface would benefit a subject greatly. The compositions provided herewith could also be used, as described below, for a variety of mucosal malignancies. Mucosal immunity generally involves immunoglobulin of the secretory IgA (s-IgA) isotype, and accordingly, antibodies of this isotype could be used together with the agents of Formula I, although such antibodies are not so limited. The agents of Formula I are useful in stimulating both cell-mediated immune responses and antibody-mediated immune responses at mucosal surfaces. Mucosal surfaces include oral, rectal, vaginal, gastrointestinal surfaces.

The novel observation that Formula I compounds induce the production of IL-1 indicates that such compounds can be used for a number of indications that are mediated fully or in part by IL-1 and downstream IL-1 signaling events. Some of these indications are recited herein as targets of combination therapy. It has been discovered according to the invention that some of these indications can also respond to sole Formula I compound administration.

Formula I compounds can be used either alone or in combination with other active agents to treat viral infections, particularly chronic infections, and more particularly chronic hepatitis C infection. Currently, hepatitis C subjects are administered IFNA, however not all subjects are treated using this therapy. Moreover, subjects that are also HIV positive fair even worse with this treatment. It has been found according to the invention that hepatitis C infected subjects, and especially those subjects resistant or non-responsive to IFNα treatment, can be treated using Formula I compounds. In some instances, the Formula I compounds can be administered with IFNα (which in turn may be in pegylated form), and optionally with ribavirin also. In these subjects, Formula I compounds can also be used together with other small molecule drugs that are currently being tested for hepatitis C infection.

The compounds of the invention are also suitable for treatment of hepatitis B infection. In this latter indication, Formula I compounds can be used alone or together with IFN as well as various small molecule drugs being developed, such as IFNα-2b, acyclovir, lobucavir, ganciclovir, L-deoxythymidine, clevudine, a therapeutic vaccine, phosphonoformate (PFA), ribavirin (RBV) and thymosin alpha-1; and nucleotide and nucleoside analogues such as 2 3-dideoxy-3-fluoroguanosine (FLG), famciclovir, lamivudine, adefovir dipivoxil, entecavir, and emtricitabine. Formula I compounds can also be used with hepatitis B-specific immunoglobulin.

The use of Formula I compounds with lamivudine is particularly interesting as lamivudine is reportedly associated with drug resistance. The combined use of Formula I compounds with lamivudine can reduce or eliminate the risk of drug resistance. Alternatively, Formula I compounds may be used in subjects already treated with lamivudine who have already demonstrated drug resistance. These latter aspects of the invention apply equally to other indications for which drug resistance has been observed or is suspected. In other instances, it may be desirable to use Formula I compounds over standard drug therapy if the drug therapy is not particularly suited to a subject or induces intolerable side effects in a patient specific manner. Other bacteria that have been associated with drug resistance include Staphylococcus aureus (resistance to penicillin), Streptococcus pneumoniae (resistance to penicillin), gonorrhea (resistance to penicillin), and Enterococcus faecium (penicillin).

Formula I compounds can also be used in the treatment of tuberculosis, either alone (i.e., as a substitute for currently available drug treatments such as antibiotic therapy), or in combination with those antibiotics.

The ability of Formula I compounds to induce cytokines, and in particular IL-1, also indicates that these compounds are useful in vaccine induced immunity, including both humoral and cell-mediated immunity. The ability to enhance cellular mediated immunity is useful, inter alia, in the treatment or prevention of viral infections, and in particular, HIV infection. As described in greater detail below, Formula I compounds can be used together with vaccines such as those to small pox virus (e.g., BVL).

Induction of IL-1 indicates that Formula I compounds can be used to activate macrophages. This in turn can be exploited to reduce plaque formation in cardiovascular disease. Plaque engulfing macrophages can be activated following Formula I compound administration.

Indications relating to immune deficiency can also be treated using Formula I compounds. These indications include congenital deficiencies, some of which are described in greater detail herein. Examples include the syndromes commonly referred to as congenital disorder of glycosylation (CDG). Another congenital indication is the immunoglobulin deficiency common variable immunodeficiency (CVID) which is characterized by low IgG and IgA, and in some instances low IgM. Subjects having CVID can present with other clinical manifestations including gastrointestinal problems, granulomatous inflammation, cutaneous features, unusual presentations of enteroviral and mycoplasma infection, an increased incidence of autoimmunity, and a predisposition to lymphoma and stomach cancer. Other congenital indications include agammaglobulinemias such as Bruton's agammaglobulinemia and congenital hypogammaglobulinemia, selective immunoglobulin A deficiency, and severe combined immunodeficiency (i.e., SCID, a T cell deficiency). Immune deficiencies that include low or no immunoglobulin production can be treated using Formula I compounds alone, and in some instances, preferably with the antibodies described herein. Other immune deficiencies include amyotrophic lateral sclerosis (ALS), systemic lupus erythematosus, rheumatoid arthritis, Hashimoto's disease, chronic immune thrombocytopenic purpura (chronic ITP), and the like.

As indicated above, Formula I compounds are therapeutically and prophylactically useful for indications which are responsive to IFN therapy. The IFN therapy may be IFNα, IFNβ, or IFNγ therapy, but is not so limited. A further example of this is multiple sclerosis. Others include tuberculosis, chronic Epstein Barr Virus (EBV) infection, and chronic hepatitis (e.g., chronic hepatitis C), viral hepatitis (e.g., hepatitis C), hepatocellular carcinoma, Kaposi's Sarcoma (AIDS-related), thick primary melanomas, and regional lymph node metastases. Examples of conditions responsive to IFNγ therapy include but are not limited to viral infections and associated diseases and cancer.

One advantage of using Formula I compounds in place of IFN therapy is that Formula I compounds are less expensive and easier to administer than IFN. These and other conditions can be immunosuppressive and therefore Formula I compounds are can be used to enhance immunity in such subjects. Other chronic immunosuppressive conditions can arise from pharmaceutical use such as the use of deliberate anti-inflammatories such as cox-1 or cox-2 inhibitors celecoxib (Celebrex), rofecoxib (Vioxx), naproxen (Naprosyn), non-steroidal anti-inflammatory drugs (NSAIDS) such as ibuprofen (Motrin, Advil), fenoprofen, indomethacin, and valdecoxib (Bextra), and aspirin; substance abuse such as the alcoholism, intravenous drug use, morphine use; chronic infections or disease states such as gingivitis, osteomyelitis, diabetes types I and II, chronic granulomas, Pneumocystis carinii pneumonia (PCP) infection, recurrent fungal/yeast infections, non-Hodgkin's lymphoma, and Kaposi's Sarcoma.

As a prophylaxis, Formula I compounds can be used to enhance immunity in a subject at risk of developing a condition that is immunologically responsive. For example, a subject may be administered a Formula I compound when it is at risk of developing the flu. As another example, a subject having or at risk of having angina may be administered a Formula I compound.

The invention therefore provides therapeutic and prophylactic methods that involve the administration of linear or cyclic Formula I compounds. In some instances and depending upon the indication being treated or prevented, the Formula I compounds are combined, preferably in pharmaceutical form, with antibodies or fragments thereof or antigens. Formula I compounds have the following structure:
PR Formula I
wherein P is a targeting group which binds to the reactive site of a post proline-cleaving enzyme, and R is a reactive group capable of reacting with a functional group in the reactive site of a post proline-cleaving enzyme. Post proline-cleaving enzymes are enzymes which have a specificity for removing Xaa-Pro or Xaa-Ala dipeptides (where Xaa represents any amino acid) from the amino terminus of polypeptides. Examples of post-proline cleaving enzymes include, but are not limited to, CD26, dipeptidyl peptidase IV (DP IV) and fibroblast activation protein (FAP).

The P targeting group can be composed of single or multiple residues of peptide or peptidomimetic nature, provided that such residues do not interfere significantly, and most preferably improve the site-specific recognition of post proline-cleaving enzyme by the agent of Formula I. In certain embodiments, the portion of the P targeting group that is involved in binding to the reactive site of a post proline-cleaving enzyme is formed of amino acids and the remaining portion of P is formed of non-amino acid components. According to the particular embodiment, P can be composed wholly of amino acid residues, wholly of non-amino acid substituents, or a coinmbination of both.

In general, the targeting group (i.e., P) is covalently coupled to the reactive group. In some embodiments, the covalent coupling occurs via a carboxyl group at the carboxyl terminal amino acid in the P group. In certain embodiments, P may be 30, 20, 10 or less than 10 residues in length.

The development of phage display libraries and chemical combinatorial libraries from which synthetic compounds can be selected which mimic the substrate of a protease permits the identification of further targeting groups to which an R group can be covalently attached to form a binding moiety which binds or associates with the reactive site of the protease and which forms a complex with a functional group in the protease reactive site. Such libraries can be screened to identify non-naturally occurring putative targeting groups by assaying protease cleavage activity in the presence and absence of the putative phage display library molecule or combinatorial library molecule and determining whether the molecule inhibits cleavage by the protease of a known substrate or of a substrate analog (e.g., a chromophoric substrate analog which is easily detectable in a spectrophotometric assay). Those phage library and/or combinatorial library molecules which exhibit inhibition of a post-prolyl cleaving enzyme then can be covalently coupled to the reactive groups disclosed herein and again tested to determine whether these novel molecules selectively bind to, a post-prolyl cleaving enzyme. In this manner, a simple, high-through-put screening assay is provided for identifying non-naturally occurring targeting groups of the invention.

P targeting groups can be synthesized from peptides or other biomolecules including but not limited to saccharides, fatty acids, sterols, isoprenoids, purines, pyrimidines, derivatives or structural analogs of the above, or combinations thereof and the like. Also envisioned in the invention is the use of targeting groups made from peptoids, random bio-oligomers (U.S. Pat. No. 5,650,489), benzodiazepines, diversomeres such as dydantoins, benzodiazepines and dipeptides, nonpeptidyl peptidomimetics with a beta-D-glucose scaffolding, oligocarbamates or peptidyl phosphonates. Many, if not all, of these compounds can be synthesized using recombinant or chemical library approaches. A vast array of candidate targeting groups can be generated from libraries of synthetic or natural compounds. The methods of the invention utilize this library technology to identify small peptides which bind to protease reactive sites. One advantage of using libraries for inhibitor identification is the facile manipulation of millions of different putative candidates of small size in small reaction volumes (i.e., in synthesis and screening reactions). Another advantage of libraries is the ability to synthesize targeting groups which might not otherwise be attainable using naturally occurring sources, particularly in the case of non-peptide moieties.

Examples of reactive groups useful in the invention include organo boronates, organo phosphonates, fluoroalkylketones, alphaketos, N-peptiolyl-O-(acylhydroxylamines), azapeptides, azetidines, fluoroolefins dipeptide isoesteres, peptidyl (alpha-aminoalkyl) phosphonate esters, aminoacyl pyrrolidine-2-nitriles and 4-cyanothiazolidides.

Some representative agents of Formula I can be further defined by Formula II as follows: 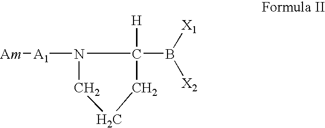
wherein m is an integer between 0 and 10, inclusive; A and A₁ may be L- or D-amino acid residues (for glycine there is no such distinction) such that each A in Aₘ may be an amino acid residue different from another or all other A in Aₘ; the C bonded to B is in the L-configuration; the bond between A₁ and N and, in some embodiments, the bond between A and A₁ are peptide bonds; and each X₁ and X₂ is, independently, a hydroxyl group or a group capable of being hydrolyzed to a hydroxyl group in aqueous solution at physiological pH. By “the C bonded to B is in the L-configuration” is meant that the absolute configuration of the C is like that of an L-amino acid.

Thus, the 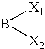
group has the same relationship to the C as the —COOH group of an L-amino acid has to its a carbon. In various embodiments, A and A₁ are independently proline or alanine residues; m is 0; X₁ and X₂ are hydroxyl groups.

One group of Formula I compounds useful in the invention can be further defined by Formula III 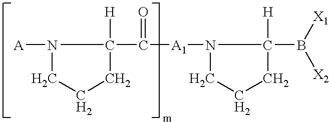
wherein m is an integer between 0 and 10, inclusive; A and A₁ are L- or D-amino acid residues (naturally or non-naturally occurring); A in each repeating bracketed unit can be a different amino acid residue; the C bonded to B is in the L-configuration; the bonds between A and N, A₁ and C, and between A₁ and N are peptide bonds; and each X₁ and X₂ is, independently, a hydroxyl group or a group capable of being hydrolyzed to a hydroxyl group in aqueous solution at physiological pH.

Thus, in one embodiment, the compound is L-Ala-L-boroPro; and the compound is L-Pro-L-boroPro. In important embodiments, the compound is Val-boroPro.

These compounds can be provided and used in linear or cyclic form, as described in U.S. Pat. No. 6,355,614, issued Mar. 12, 2002.

Other agents useful in the methods and compositions of the invention are derivatives of Formula II in which each and every A in Aₘ may independently be a non-amino acid residue. Thus, the plurality of A (i.e., Aₘ, wherein m>1) may be a peptide or a peptidomimetic which may include, in whole or in part, non-amino acid residues such as saccharides, fatty acids, sterols, isoprenoids, purines, pyrimidines, derivatives or structural analogs of the above, or combinations thereof and the like. The plurality of A in Aₘ may also be comprised of a combination of amino acid and non-amino acid residues. It also is possible to substitute non-naturally occurring amino acids, such as 2-azetidinecarboxylic acid or pipecolic acid (which have 6-membered, and 4-membered ring structures respectively) for the proline residue. Representative structures of transition-state analog-based inhibitors Xaa-boroPro of Formula II, include Lys-BoroPro, Pro-BoroPro and Ala-BoroPro in which “boroPro” refers to the analog of proline in which the carboxylate group (COOH) is replaced with a boronyl group [B(OH)₂]. Alternative compounds of the invention have an analogous structure in which the boronyl group is replaced by, for example, a phosphonate or a fluoroalkylketone, alphaketos, N-peptiolyl-O-(acylhydroxylamines), azapeptides, azetidines, fluoroolefins dipeptide isoesteres, peptidyl (alpha-aminoalkyl) phosphonate esters, aminoacyl pyrrolidine-2-nitriles and 4-cyanothiazolidides. It is to be understood that each and every reactive group described herein can be substituted for the reactive group of Formula II (i.e., boronyl group). Where appropriate these limitations apply equally to Formula III compounds.

All amino acids, with the exception of glycine, contain an asymmetric or chiral carbon and may contain more than one chiral carbon atom. The asymmetric a carbon atom of the amino acid is referred to as a chiral center and can occur in two different isomeric forms. These forms are identical in all chemical and physical properties with one exception, the direction in which they can cause the rotation of plane-polarized light. These amino acids are referred to as being “optically active,” i.e., the amino acids can rotate the plane-polarized light in one direction or the other.

The four different substituent groups attached to the a carbon can occupy two different arrangements in space. These arrangements are not superimposable mirror images of each other and are referred to as optical isomers, enantiomers, or stereo isomers. A solution of one stereo isomer of a given amino acid will rotate plane polarized light to the left and is called the levorotatory isomer [designated (−)]; the other stereo isomer for the amino acid will rotate plane polarized light to the same extent but to the right and is called dextrorotatory isomer [designated (+)].

A more systematic method for classifying and naming stereo isomers is the absolute configuration of the four different substituents in the tetrahedron around the asymmetric carbon atom (e.g., the a carbon atom). To establish this system, a reference compound was selected (glyceraldehyde), which is the smallest sugar to have an asymmetric carbon atom. By convention in the art, the two stereo isomers of glyceraldehyde are designated L and D. Their absolute configurations have been established by x-ray analysis. The designations, L and D, also have been assigned to the amino acids by reference to the absolute configuration of glyceraldehyde. Thus, the stereo isomers of chiral compounds having a configuration related to that of L-glyceraldehyde are designed L, and the stereo isomers having a configuration related to D-glyceraldehyde are designated D, regardless of the direction in which they rotate the plane-polarized light. Thus, the symbols, L and D, refer to the absolute configuration of the four substituents around the chiral carbon.

In general, naturally occurring compounds which contain a chiral center are only in one stereo isomeric form, either D or L. The naturally occurring amino acids are the L stereo isomers; however, the invention embraces amino acids which can be in the D stereo isomer configuration.

Most amino acids that are found in proteins can be unambiguously named using the D L system. However, compounds which have two or more chiral centers may be in 2ⁿ possible stereo isomer configurations, where n is the number of chiral centers. These stereo isomers sometimes are designated using the RS system to more clearly specify the configurations of amino acids that contain two or more chiral centers. For example, compounds such as threonine isoleucine contain two asymmetric carbon atoms and therefore have four stereo isomer configurations. The isomers of compounds having two chiral centers are known as diastereomers. A complete discussion of the RS system of designating optical isomers for amino acids is provided in Principles in Biochemistry, editor A. L. Lehninger, page 99-100, supra. A brief summary of this system follows.

The RS system was invented to avoid ambiguities when a compound contains two or more chiral centers. In general, the system is designed to rank the four different substituent atoms around an asymmetric carbon atom in order of decreasing atomic number or in order of decreasing valance density when the smallest or lowest-rank group is pointing directly away from the viewer. The different rankings are well known in the art and are described on page 99 of Lehninger (supra). If the decreasing rank order is seen to be clock-wise, the configuration around the chiral center is referred to as R; if the decreasing rank order is counter-clockwise, the configuration is referred to as S. Each chiral center is named accordingly using this system. Applying this system to threonine, one skilled in the art would determine that the designation, L-threonine, refers to (2S, 3R)-threonine in the RS system. The more traditional designations of L-, D-, L-allo, and D-allo, for threonine have been in common use for some time and continue to be used by those of skill in this art. However, the R S system increasingly is used to designate the amino acids, particularly those which contain more than one chiral center.

The agents of the invention may be in some instances substantially optically pure. That is, at least 90%, 92%, 94%, 95%, 96%, 97%, 98% or 99% of the carbon atoms bearing boron are of the L-configuration in some embodiments. Methods for synthesizing optically pure isomers of Formula I agents are disclosed in published PCT application WO 93/08259.

Many of the agents of the invention and methods for their manufacture have been previously disclosed in U.S. Pat. No. 4,935,493, the contents of which are incorporated by reference herein.

As mentioned earlier, the agents, including their individual targeting and reactive groups, may be synthesized using recombinant or chemical library synthesis approaches. Libraries of interest in the invention include peptide libraries, synthetic organic combinatorial libraries, and the like. The artisan of ordinary skill is familiar with the methodology for library and combinatorial chemistry synthesis as well as the screening of such compounds for agents which are useful in the methods of the invention. The use of library technology, such as phage display, and combinatorial chemistry, such as compound array methods, in the synthesis and screening of protease inhibitors has been previously described in U.S. patent application entitled “Multivalent Compounds for Crosslinking Receptors and Uses Thereof” filed on Apr. 12, 1999 and assigned U.S. Ser. No. 09/290,376, the contents of which are incorporated in their entirety by reference. Examples of parallel synthesis mixtures and parallel synthesis methods are provided in U.S. Ser. No. 08/177,497, filed Jan. 5, 1994 and its corresponding PCT published patent application W095/18972, published Jul. 13, 1995 and U.S. Pat. No. 5,712,171 granted Jan. 27, 1998 and its corresponding PCT published patent application W096/22529, which are hereby incorporated by reference.

Certain methods and compositions comprise, in addition to the compounds of Formula I, an antibody or fragment thereof. The invention embraces the use of antibodies of all isotypes including IgM, IgA1, IgA2, sigA, IgD, IgE, IgG1, IgG2, IgG3, and IgG4, having light chains that are either kappa or lambda chains.

The antibodies or fragments thereof useful in the invention can be specific for any component of a particular target. Accordingly, the antibody can recognize and bind to proteins, lipids, carbohydrates, DNA, RNA, and any combination of these in molecular or supra-molecular structures (e.g., cell organelles such as mitochondria or ribosomes). The antibody or fragment thereof can also recognize a modification of the tumor cell, such as e.g., chemical modifications, or genetic modifications made by transfection ex vivo or in vivo with DNA or RNA. As used herein, the terms “antibody” and “immunoglobulin” are used interchangeably.

Bispecific antibodies can also be used in the invention. A bispecific antibody is one having one variable region that specifically recognizes a tumor antigen and the other variable region that specifically recognizes an antigenic epitope of a host immune effector cell that has lytic or growth inhibitory activity against the tumor. Bispecific and multispecific antibody complexes can be created by linkage of two or more immunoglobulins of different specificity for tumor antigens and/or effector cell antigens, either at the peptide or nucleic acid level.

Immunoglobulin can be produced in vivo in human or nonhuman species, or in vitro from immunoglobulin encoding DNA or cDNA isolated from libraries of DNA (e.g., phage display libraries). Immunoglobulin can also be modified genetically or chemically to incorporate human polypeptide sequences into non-human coding sequences (commonly referred to as humanization). Additionally, immunoglobulins can be modified chemically or genetically to incorporate protein, lipid, or carbohydrate moieties. Potential modifications could also include naturally occurring or synthetic molecular entities that are either directly toxic for tumor cells or serve as ligands or receptors for biologically active molecules that could suppress tumor growth. For example, growth factors, cytokines, chemokines and their respective receptors, immunologically active ligands or receptors, hormones or naturally occurring or synthetic toxins all represent biologically active molecules that could interact with suitably modified immunoglobulins and their targets.

The antibody or antibody fragment may conjugated (covalently or otherwise) to a toxin derived from plant, fungus, or bacteria. The toxin may be selected from the group consisting of A chain toxin, deglycosylated A chain toxin, ribosome inactivating protein, α-sarcin, aspergillin, restrictocin, ribonuclease, diptheria toxin and Pseudomonas exotoxin, but is not so limited.

The antibody or antibody fragment may also conjugated to a chemotherapeutic agent, a radioisotope such as those recited herein, or a cytotoxin. The chemotherapeutic agent may be selected from the group consisting of an anti-metabolite, an anthracycline, a vinca alkaloid, an antibiotic, an alkylating agent, and an epipodophyllotoxin, but is not so limited.

As used herein, an “anti-cancer antibody or fragment thereof” is an antibody or an antibody fragment that binds to a cancer or tumor antigen. The terms “cancer antigen” and “tumor antigen” are used interchangeably. A cancer antigen as used herein is a compound differentially associated with a tumor or cancer, preferably at the cell surface of a tumor or cancer cell, that is capable of invoking an immune response. The cancer antigen may be peptide in nature but it is not so limited. As an example, the antigen may be a lipid antigen, as described in U.S. Pat. No. 5,679,347, issued on Oct. 21, 1997 and U.S. Pat. No. 6,238,676 B1, issued on May 29, 2001. If the antigen is a peptide, then it invokes an immune response when it is presented (in a digested form) on the surface of an antigen presenting cell in the context of an MHC molecule. If the antigen is a lipid, then it invokes an immune response when it is presented in the context of a CD1 molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A cancer antigen encompasses antigens that are differentially expressed between cancer and normal cells. Due to this differential expression, these antigens can be targeted in anti-tumor therapies. Cancer antigens may be expressed in a regulated manner in normal cells. For example, they may be expressed only at certain stages of differentiation or at certain points in development of the organism or cell. Some are temporally expressed as embryonic and fetal antigens. Still others are never expressed in normal cells, or their expression in such cells is so low as to be undetectable.

Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

Examples of cancer antigens include HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Other cancer antigens are described in U.S. Pat. No. 5,776,427. Still other cancer antigens are listed in Table 1.

Further examples include MAGE, MART-1/Melan-A, gp100, Dipeptidyl peptidase IV (DPPIV), adenosine deaminase-binding protein (ADAbp), FAP, cyclophilin b, Colorectal associated antigen (CRC)-C017-1A/GA733, Carcinoembryonic Antigen (CEA) and its immunogenic epitopes CAP-1 and CAP-2, etv6, am11, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pme117}, PRAME, NY-ESO-1, cdc27, adenomatous polyposis coli protein (APC), fodrin, Connexin 37, Ig-idiotype, p 15, gp75, GM2 and GD2 gangliosides, viral products such as human papilloma virus proteins, Smad family of tumor antigens, lmp-1, PIA, EBV-encoded nuclear antigen (EBNA)-1, brain glycogen phosphorylase, SSX-1, SSX-2 (HOM-MEL-40), SSX-1, SSX-4, SSX-5, SCP-I and CT-7, CD20 and c-erbB-2.

These antigens can be classified as indicated in Tables 1.

TABLE 1
Classification of cancer antigens
Table 1a. Proteins encoded by genes that have undergone
chromosomal alteration in lymphoma and leukemia

| Genes | Disease |
|---|---|
| Activation of quiescent genes | |
| BCL-1 and IgH | Mantel cell lymphoma |
| BCL-2 and IgH | Follicular lymphoma |
| BCL-6 | Diffuse large B-cell lymphoma |
| TAL-1 and TCRδ or SIL | T-cell acute lymphoblastic leukemia |
| c-MYC and IgH or IgL | Burkitt lymphoma |
| MUN/IRF4 and IgH | Myeloma |
| PAX-5 (BSAP) | Immunocytoma |
| Creation of fusion genes | |
| RARα, PML, PLZF, NPM or NuMA | Acute promyelocytic leukemia |
| BCR and ABL | Chronic myeloid/acute lymphoblastic leukemia |
| MLL (HRX) | Acute leukemia |
| E2A and PBX or HLF | B-cell acute lymphoblastic leukemia |
| NPM, ALK | Anaplastic large cell leukemia |
| NPM, MLF-1 | Myelodysplastic syndrome/acute myeloid |
| | leukemia |

Adapted from Falini B. and Mason, D.Y. (2002) Blood 99: 409-426

Table 1b. Proteins specific to a tissue or cell lineage

| Protein | Disease |
|---|---|
| Cell-surface proteins | |
| CD20, CD22 | Non-Hodgkin's lymphoma, B-cell lymphoma, |
| | Chronic lymphocytic leukemia (CLL) |
| CD52 | B-cell CLL |
| CD33 | Acute myelogenous leukemia (AML) |
| CD10 (gp100) | Common (pre-B) acute lymphocytic leukemia |
| | and malignant melanoma |
| CD3/T-cell receptor (TCR) | T-cell lymphoma and leukemia |
| CD79/B-cell receptor (BCR) | B-cell lymphoma and leukemia |
| CD26 | Epithelial and lymphoid malignancies |
| Human leukocyte antigen (HLA)-DR, HLA- | Lymphoid malignancies |
| DP, and HLA-DQ | |
| RCAS1 | Gynecological carcinomas, bilary |
| | adenocarcinomas and ductal adenocarcinomas |
| | of the pancreas |
| Prostate specific membrane antigen | Prostate cancer |
| Epidermal growth factor receptors (high | |
| expression | |
| EGFR (HER1 or erbB1) and EGFRvIII | Brain, lung, breast, prostate and stomach |
| | cancer |
| erbB2 (HER2 or HER2/neu) | Breast cancer and gastric cancer |
| erbB3 (HER3) | Adenocarcinoma |
| erbB4 (HER4) | Breast cancer |
| Cell-associated proteins | |
| Tyrosinase, Melan-A/MART-1, tyrosinase | Malignant melanoma |
| related protein (TRP)-1/gp75 | |
| Polymorphic epithelial mucin (PEM) | Breast tumors |
| Human epithelial mucin (MUC1) | Breast, ovarian, colon and lung cancers |
| Secreted proteins | |
| Monoclonal immunoglobulin | Multiple myeloma and plasmacytoma |
| Immunoglobulin light chains | Multiple Myeloma |
| α-fetoprotein | Liver carcinoma |
| Kallikreins 6 and 10 | Ovarian cancer |
| Gastrin-releasing peptide/bombesin | Lung carcinoma |
| Prostate specific antigen | Prostate cancer |

Table 1c. Cancer testis (CT) antigens*

These antigens include MAGE-A1, -A3, -A6, -A12, BAGE, GAGE, HAGE, LAGE-1, NY-ESO-1,
RAGE, SSX-1, -2, -3, -4, -5, -6, -7, -8, -9, HOM-TES-14/SCP-1, HOM-TES-85 and PRAME.
*These antigens are expressed in some normal tissues such as testis and in some cases placenta. Their
expression is common in tumors of diverse lineages and as a group the antigens form targets for
immunotherapy. Examples of tumor expression of CT antigens are as follows.

| Protein | Disease |
|---|---|
| SSX-2, and -4 | Neuroblastoma |
| SSX-2 (HOM-MEL-40), MAGE, GAGE, BAGE | Malignant melanoma |
| and PRAME | |
| HOM-TES-14/SCP-1 | Meningioma |
| SSX-4 | Oligodendrioglioma |
| HOM-TES-14/SCP-1, MAGE-3 and SSX-4 | Astrocytoma |
| SSX member | Head and neck cancer, ovarian cancer, lymphoid |
| | tumors, colorectal cancer and breast cancer |
| RAGE-1, -2, -4, GAGE-1, -2, -3, -4, -5, -6, -7 and | Head and neck squamous cell carcinoma |
| -8 | (HNSCC) |
| HOM-TES14/SCP-1, SSX-1, PRAME and CT-7 | Non-Hodgkin's lymphoma |
| PRAME | Acute lymphoblastic leukemia (ALL), acute |
| | myelogenous leukemia (AML) and chronic |
| | lymphocytic leukemia (CLL) |

Table 1d. Proteins not-specific to a tissue or cell lineage*

Carcinoembryonic antigen (CEA) family: CD66a, CD66b, CD66c, CD66d and CD66e.
*These antigens can be expressed in many different malignant tumors and can be targeted by
immunotherapy.

Table 1e. Viral proteins

Human papilloma virus protein (cervical cancer)
EBV-encoded nuclear antigen (EBNA)-1 (lymphomas of neck and oral cancer)

Table 1f. Mutated or aberrantly expressed molecules

CDK4 and beta-catenin in melanoma

Cancer or tumor antigens can also be classified according to the cancer or tumor they are associated with (i.e., expressed by). Cancers or tumors associated with tumor antigens include acute lymphoblastic leukemia (etv6; am11; cyclophilin b), B cell lymphoma (Ig-idiotype); Burkitt's (Non-Hodgkin's) lymphoma (CD20); glioma (E-cadherin; α-catenin; β-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)-C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkin's lymphoma (lmp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21 ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins and non-infectious particles), testicular cancer (NY-ESO-1), T cell leukemia (HTLV-1 epitopes), and melanoma (Melan-A/MART-1; cdc27; MAGE-3; p21ras; gp100^{Pme117}).

For examples of tumor antigens which bind to either or both MHC class I and MHC class II molecules, see the following references: Coulie, Stem Cells 13:393-403, 1995; Traversari et al., J. Exp. Med. 176:1453-1457, 1992; Chaux et al., J. Immunol. 163:2928-2936, 1999; Fujie et al., Int. J. Cancer 80:169-172, 1999; Tanzarella et al., Cancer Res. 59:2668-2674, 1999; van der Bruggen et al., Eur. J. Immunol. 24:2134-2140, 1994; Chaux et al., J. Exp. Med. 189:767-778, 1999; Kawashima et al, Hum. Immunol. 59:1-14, 1998; Tahara et al., Clin. Cancer Res. 5:2236-2241, 1999; Gaugler et al., J. Exp. Med. 179:921-930, 1994; van der Bruggen et al., Eur. J. Immunol. 24:3038-3043, 1994; Tanaka et al., Cancer Res. 57:4465-4468, 1997; Oiso et al., Int. J. Cancer 81:387-394, 1999; Herman et al., Innumogenetics 43:377-383, 1996; Manici et al., J. Exp. Med. 189:871-876, 1999; Duffour et al., Eur. J. Immunol. 29:3329-3337, 1999; Zorn et al., Eur. J. Immunol. 29:602-607, 1999; Huang et al., J. Immunol.162:6849-6854, 1999; Boel et al., Immunity 2:167-175, 1995; Van den Eynde et al., J. Exp. Med. 182:689-698, 1995; De Backer et al., Cancer Res. 59:3157-3165, 1999; Jager et al., J. Exp. Med. 187:265-270, 1998; Wang et al., J. Immunol. 161:3596-3606, 1998; Aarnoudse et al., Int. J. Cancer 82:442-448, 1999; Guilloux et al.,J. Exp. Med. 183:1173-1183, 1996; Lupetti et al.,J. Exp. Med. 188:1005-1016, 1998; Wolfel et al., Eur. J. Immunol. 24:759-764, 1994; Skipper et al., J. Exp. Med. 183:527-534, 1996; Kang et al.,J. Immunol. 155:1343-1348, 1995; Morel et al., Int. J. Cancer 83:755-759, 1999; Brichard et al., Eur. J. Immunol. 26:224-230, 1996; Kittlesen et al., J. Immunol. 160:2099-2106, 1998; Kawakami et al., J. Immunol. 161:6985-6992, 1998; Topalian et al., J. Exp. Med. 183:1965-1971, 1996; Kobayashi et al., Cancer Research 58:296-301, 1998; Kawakami et al.,J. Immunol. 154:3961-3968, 1995; Tsai et al., J. Immunol. 158:1796-1802, 1997; Cox et al., Science 264:716-719, 1994; Kawakami et al., Proc. Natl. Acad. Sci. USA 91:6458-6462, 1994; Skipper et al., J. Immunol. 157:5027-5033, 1996; Robbins et al., J. Immunol. 159:303-308, 1997; Castelli et al, J. Immunol. 162:1739-1748, 1999; Kawakami et al.,J. Exp. Med. 180:347-352, 1994; Castelli et al.,J. Exp. Med. 181:363-368, 1995; Schneider et al., Int. J. Cancer 75:451-458, 1998; Wang et al., J. Exp. Med 183:1131-1140, 1996; Wanget al.,J. Exp. Med. 184:2207-2216, 1996; Parkhurstet al., Cancer Research 58:4895-4901, 1998; Tsang et al., J. Natl Cancer Inst 87:982-990, 1995; Correale et al., J. Natl Cancer Inst 89:293-300, 1997; Coulie et al., Proc. Natl. Acad. Sci. USA 92:7976-7980, 1995; Wolfel et al., Science 269:1281-1284, 1995; Robbins et al., J. Exp. Med. 183:1185-1192, 1996; Brandle et al., J. Exp. Med. 183:2501-2508, 1996; ten Bosch et al., Blood 88:3522-3527, 1996; Mandruzzato et al., J. Exp. Med. 186:785-793, 1997; Gueguen et al., J. Immunol. 160:6188-6194, 1998; Gjertsen et al., Int. J. Cancer 72:784-790, 1997; Gaudin et al., J. Immunol. 162:1730-1738, 1999; Chiari et al., Cancer Res. 59:5785-5792, 1999; Hogan et al., Cancer Res. 58:5144-5150, 1998; Pieper et al., J. Exp. Med. 189:757-765, 1999; Wang et al., Science 284:1351-1354, 1999; Fisk et al., J. Exp. Med. 181:2109-2117, 1995; Brossart et al., Cancer Res. 58:732-736, 1998; Ropke et al., Proc. Natl. Acad. Sci. USA 93:14704-14707, 1996; Ikeda et al., Immunity 6:199-208, 1997; Ronsin et al., J. Immunol. 163:483-490, 1999; Vonderheide et al., Immunity 10:673-679,1999. These antigens as well as others are disclosed in PCT Application PCT/US98/18601.

In some embodiments, the antigens are administered in a substantially purified form. The term “substantially purified” as used herein refers to a compound which is substantially free of other compounds such as proteins, lipids, carbohydrates or other materials with which it is naturally associated. One skilled in the art can purify viral or bacterial compounds such as polypeptides using standard techniques such as for example protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis.

The Formula I compounds can be used in combination with various vaccines either currently being used or in development, whether intended for human or non-human subjects. Examples of vaccines for human subjects and directed to infectious diseases include the combined diphtheria and tetanus toxoids vaccine; pertussis whole cell vaccine; the inactivated influenza vaccine; the 23-valent pneumococcal vaccine; the live measles vaccine; the live mumps vaccine; live rubella vaccine; Bacille Calmette-Guerin (BCG) tuberculosis vaccine; hepatitis A vaccine; hepatitis B vaccine; hepatitis C vaccine; rabies vaccine (e.g., human diploid cell vaccine); inactivated polio vaccine; meningococcal polysaccharide vaccine; quadrivalent meningococcal vaccine; yellow fever live virus vaccine; typhoid killed whole cell vaccine; cholera vaccine; Japanese B encephalitis killed virus vaccine; adenovirus vaccine; cytomegalovirus vaccine; rotavirus vaccine; varicella vaccine; anthrax vaccine; small pox vaccine.

The compounds of Formula I could be administered after viral, bacterial mycobacterial, fungal, or parasitic infection in order to stimulate innate immunity (i.e., immunity mediated by neutrophils, macrophages, NK cells and eosinophils) and/or adaptive immunity (i.e., immunity mediated by T cells and B cells). The growth factors, cytokines and chemokines stimulated by the compounds of Formula I (e.g., Val-boroPro (PT-100)) can stimulate these cells and thereby enhance an immune response to a foreign pathogen. As an example, IL-1β rapidly activates innate immunity. Therefore, Formula I compounds can be used to activate innate immunity via IL-1β induction, and this in turn can provide an initial defense against any infectious agent.

The compounds of Formula I can also be used prophylactically to prevent infection during periods of heightened risk, including for example flu season, epidemics, and travel to places where the risk of pathogen exposure is high. Many of the cytokines and chemokines induced by Formula I compounds can prime a subject and prepare it for passive exposure to a pathogen. The rate at which Formula I compounds stimulate these cytokines and chemokines (e.g., IL-1β) is useful particularly where pathogen exposure cannot be anticipated.

Thus, the methods of the invention can be used in the treatment or prevention of infectious diseases such as bacterial infections, mycobacterial infections, viral infections, fungal infections and parasitic infections.

Examples of bacterial infections include E. coli, Streptococcal infections, Staphylococcal infections, Pseudomonas infections, Clostridium difficile, Legionella infections, Pneumococcus infection, Haemophilus infections (e.g., Haemophilus influenzae infections), Klebsiella infections, Enterobacter infections, Citrobacter infections, Neisseria infections (e.g., N. meningitidis infection, N. gonorrhoeae infection), Shigella infections, Salmonella infections, Listeria infections (e.g., L. monocytogenes infection), Pasteurella infection (e.g., Pasteurella multocida infection), Streptobacillus infection, Spirillum infection, Treponema infection (e.g., Treponema pallidum infection), Actinomyces infection (e.g., Actinomyces israelli infection), Borrelia infection, Corynebacterium infection, Nocardia infection, Gardnerella infections (e.g., Gardnerella vaginal is infection), Campylobacter infections (e.g., Campylobacter fetus infection), Spirochaeta infections, Proteus infections, Bacteriodes infections, H. pylori, and anthrax.

Examples of viral infections include HIV infection, Herpes simplex virus 1 and 2 infections (including encephalitis, neonatal and genital forms), human papilloma virus infection, cytomegalovirus infection, Epstein Barr virus infection, Hepatitis virus A, B and C infections, rotavirus infection, adenovirus infection, influenza A virus infection, respiratory syncytial virus infection, varicella-zoster virus infections, small pox infection, monkey pox infection and SARS infection.

Examples of fungal infections include candidiasis infection, ringworm, histoplasmosis infection, blastomycosis infections, paracoccidioidomycosis infections, crytococcosis infections, aspergillosis infections, chromomycosis infections, mycetomna infections, pseudallescheriasis infection, and tinea versicolor infection.

Examples of parasite infections include both protozoan infections and nematode infections. These include amebiasis, Trypanosoma cruzi infection (i.e., Chagas' disease), Fascioliasis (e.g., Facioloa hepatica infection), Leishmaniasis, Plasmodium infections (e.g., malaria causing Plasmodium species infections, e.g., P. falciparum, P. knowlesi, P. malariae,) Onchocerciasis, Paragonimiasis, Trypanosoma brucei infection (i.e., Sleeping sickness), Pneumocystis infection (e.g., Pneumocystis carinii infection), Trichomonas vaginalis infection, Taenia infections, Hymenolepsis infections (e.g., Hymenolepsis nana infection), Echinococcus infections, Schistosomiasis (e.g., Schistosoma mansoni infection), neurocysticercosis, Necator americanus infection, and Trichuris trichuria infections.

Other infections that can be treated according to the methods of the invention include Chlamydia infection, Mycobacterial infection such as tuberculosis and leprosy, and Rickettsiae.

The foregoing lists of infections are not intended to be exhaustive but rather exemplary. Those of ordinary skill in the art will identify other infections that are amenable to prevention and treatment using the methods of the invention.

Antigens associated with infectious diseases that can be used in the methods of the invention include whole bacteria, whole virus, whole fungi, whole parasites, and fragments thereof. Examples include non-infectious human papillomavirus-like particles (VLP) (which can be used as a cancer antigen as well, particularly for cervical cancer); and the like.

Subjects having an infectious disease are those that exhibit symptoms of infectious disease (e.g., rapid onset, fever, chills, myalgia, photophobia, pharyngitis, acute lymphadenopathy, splenomegaly, gastrointestinal upset, leukocytosis or leukopenia) and in whom infectious pathogens or byproducts thereof can be detected. Tests for diagnosing infectious diseases are known in the art and the ordinary medical practitioner will be familiar with these laboratory tests which include but are not limited to microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner will generally also take a full history and conduct a complete physical examination in addition to running the laboratory tests listed above.

A subject at risk of developing an infectious disease is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery, etc.

Formula I compounds are also indicated for treatment of human papillomavirus (HPV) infection. The current therapy for HPV is injection of IFN into a lesion and/or surgical ablation. A systemic treatment such as that envisioned for Formula I compounds, particularly when administered orally, would be desirable in comparison with current clinical therapies. Formula I compounds are similarly useful in combination with HPV vaccines currently in development such as HPV virus-like particle (VLP)-based vaccine (see, for example, Virology 2000 Jan. 20;266(2):237-45).

In still further aspects, the invention contemplates the use of Formula I compounds together with anti-microbial agents (e.g., anti-bacterial agents or anti-viral agents) in order to reduce the risk of drug resistance by the microbial species, or for treatment following incidence of drug resistance.

The invention intends to treat subjects that are not immunocompromised in some instances. Subject that are not immunocompromised (i.e., “non-immunocompromised”) are those that have blood cell counts in the normal range. Normal ranges of blood counts are known to the medical practitioner and reference can be made to a standard hematology textbook for such counts. In addition, reference can be made to published PCT application PCT/US00/14505. Non-immunocompromised subjects can include subjects that have not undergone any treatment that would render them immunocompromised. For example, such subjects may have a cancer but they have not undergone any treatment such as chemotherapy or radiation that would render them immunocompromised. Such subjects also would not inherently be immunocompromised as a result of the cancer. In some important embodiments, the subjects are at risk of developing an infection due to an impending surgical procedure, travel to a region where one or more infections are common, or they have experienced a skin abrasion, for example as a result of a trauma.

In still other embodiments, the subjects may be genetically immunocompromised, meaning that they harbor a genetic mutation that renders them immunocompromised even in the absence of an infectious or exogenous procedure. Such subjects may have for example a genetic mutation such as in agammaglobulinemia or SCID. These subjects may be treated according to the invention routinely or only when they are at a higher risk of developing an infectious disease e.g., when traveling to a region where infections are common, when having surgery, when having a skin abrasion, etc.

In still other embodiments, the methods taught herein are intended for use in elderly subjects. As used herein, an elderly subject is one that is at least 50 years old, preferably at least 60 years old, more preferably at least 70 years old, and most preferably at least 75 years old.

In some embodiments, the compositions provided herein can further include other therapeutic agents such as antimicrobials agents, if the disease is an infectious disease, or anti-cancer agents if the disease is a cancer. Examples of anti-microbials include anti-bacterials, anti-mycobacterials, anti-virals, anti-fungal, and anti-parasites.

Examples of anti-bacterials include β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams,), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines.

Anti-bacterials include: Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexii Hydrochloride; Cephaloglycini; Cephaloridine; Cephalothin Sodium; Cephapirin Sodium; Cephradine; Cetocycline Hydrochloride; Cetophenicol; Chloramphenicol; Cliloramphenicol Palmitate; Chloramphenicol Pantotheniate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clildamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycine; Demeclocycine Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Anti-mycobacterials include Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate and Trecator-SC (Ethionamide).

Anti-virals include amantidine and rimantadine, ribivarin, acyclovir, vidarabine, trifluorothymidine, ganciclovir, zidovudine, retinovir, and interferons.

Anti-virals further include: Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Soinantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime and integrase inhibitors.

Anti-fungals include imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhbitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); sulfated naphthylamine (e.g., suramin).

Other anti-infectives include Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aininacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal: Troclosene Potassium.

The antibodies that can be used with the compounds of Formula I include those useful in cancer and infectious disease as well as other disorders for which antibodies and antigens have been identified and which would benefit from an enhanced immune response.

The Formula I compounds can also be used with normal and hyper-immune globulin therapy. Normal immune globulin therapy utilizes an antibody product from the serum of normal blood donors. This pooled product contains low titers of antibody to a wide range of antigens such as those of infectious pathogens (e.g., bacteria, viruses such as hepatitis A, parvovirus, enterovirus, fungi and parasites). Hyper-immune globulin therapy utilizes antibodies which are prepared from the serum of individuals who have high titers of an antibody to a particular antigen. The antibodies may be those that are currently used or in development for treating infectious diseases. Examples include zoster immune globulin (useful for the prevention of varicella-zoster in immunocompromised children and neonates), human rabies immunoglobulin (useful in the post-exposure prophylaxis of a subject bitten by a rabid animal), hepatitis A or B immune globulin (useful in the prevention of hepatitis A or B virus, especially in a subject exposed to the virus), RSV immune globulin (useful in the treatment of respiratory syncitial virus infections), tetanus immunoglobulin; measles immunoglobulin (useful in the prevention of infection in immunocompromised or adult subjects); rubella immunoglobulin (useful in the prevention of infection in pregnant female subjects).

Other antibodies for infectious diseases include anti-shiga toxin antibodies, anti-staphylococcal antibodies (Virion Systems), and the like.

Antibodies specific for CD20 include Rituxan™, IDEC-Y2B8. Antibodies specific for HER2/neu include Herceptin™.

Some commercially available anti-cancer antibodies along with their commercial source are as follows: anti-CD20 mAb (monoclonal antibody), rituximab, Rituxan™, Non-Hodgkin's lymphoma, B cell lymphoma (IDEC/Genentech); anti-CD20 mAb, tositumomab Bexxar, Non-Hodgkin's lymphoma (Corixa/GlaxoSmithKline); anti-HER2, trastuzumab, Herceptin™, breast and ovarian cancer (Genentech); anti-HER2, MDX-210, prostate, non-small cell lung cancer, breast, pancreatic, ovarian, renal and colon cancer (Medarex/Novartis); anti-CA 125 mAb, oregovoinab, B43.13, Ovarex™, ovarian cancer (Altarex); Breva-Rex, multiple myeloma, breast, lung, ovarian (Altarex); AR54, ovarian, brest, lung (Altarex); GivaRex, pancreas, stomach, colorectal (Altarex); ProstaRex, prostate (Altarex); anti-EGF receptor mAb, IMC-C225, Erbitux™, breast, head and neck, non-small cell lung, renal, prostate, colorectal and pancreatic cancer (ImClone Systems); anti-EGF receptor mAb, MDX-447, head and neck, prostate, lung, bladder, cervical, ovarian cancer (Medarex/Merck); gemtuzumab ozogamicin, Mylotarg, CMA-676, anti-CD33 (Wyeth Pharmaceuticals); anti-tissue factor protein (TF), (Sunol); ior-c5, colorectal cancer; ceal, colorectal cancer; c5, colorectal cancer; anti-EGF receptor mAb, MDX-447, head and neck, prostate, lung, bladder, cervical and ovarian cancer (Medarex/Merck); anti-17-1A mAb, edrecolomab, Panorex, colorectal, pancreatic, lung, breast and ovarian cancer (Centocor/Glaxo/Ajinomoto); anti-CD20 mAb (Y-90 labeled), ibritumomab tiuxetan (IDEC-Y2B8), Zevalin, Non-Hodgkin's lymphoma (IDEC); anti-idiotypic mAb mimic of ganglioside GD3 epitope, BEC2, small cell lung carcinoma, melanoma (imClone Systems); anti-HLA-Dr10 mAb (1311 LYM-1), Oncoly™, Non-Hodgkin's lymphoma (Peregrine Pharmaceuticals); anti-CD33 humanized mAb (SMART M195), Zamyl™, acute myeloid leukemia, acute promyelocytic leukemia (Protein Design Labs); anti-CD52 humAb (LDP-03), CAMPATH, chronic lymphocytic leukemia (Millenium Pharmaceuticals/Ilex Oncology); anti-CD1 mAb, ior t6, cancer (Center of Molecular Immunology); anti-CAR (complement activating receptor) mAb, MDX-11, myeloid leukemia (Medarex); humanized bispecific mAb conjugates (complement cascade activators), MDX-22, myeloid leukemia (Medarex); OV 103 (Y-90 labeled antibody), celogovab, OncoScint™, ovarian and prostate cancer (Cytogen); anti-17-1A mAb, 3622W94, non-small cell lung carcinoma, prostate cancer (Glaxo Wellcome plc); anti-VEGF (RhumAb-VEGF), bevacizumab, Avastin™, lung, breast, prostate, renal and colorectal cancer (Genentech); anti-TAC (IL-2 receptor) humanized Ab (SMART), daclizumab, Zenapax, leukemia, lymphoma (Protein Design Labs); anti-TAG-72 partially humanized bispecific Ab, MDX-220, lung, colon, prostate, ovarian, endometrial, pancreatic and gastric cancer (Medarex); anti-idiotypic mAb mimic of high molecular weight proteoglycan (I-Mel-1), MELIMMUNE-1, melanoma (IDEC); anti-idiotypic mAb mimic of high molecular weight proteoglycan (I-Mel-2), MELIMMUNE-2, melanoma (IDEC); anti-CEA Ab (hMN14), CEACide™, colorectal cancer and other cancers (Immunomedics); Pretarget™ radioactive targeting agents, cancer (NeoRx); hmAbH11 scFv fragment (NovomAb-G2), H11 scFv, cancer (Viventia Biotech); anti-DNA or DNA-associated proteins (histones) mAb and conjugates, TNT (e.g. Cotara™), cancer (Peregrine Pharmaceuticals); Gliomab-H mAb, brain cancer, melanoma, neuroblastoma (Viventia Biotech); GNI-250 mAb, colorectal cancer (Wyeth); anti-EGF receptor mAb, EMD-72000, cancer (Merck KgaA); anti-CD22 humanized Ab, LymphoCide, Non-Hodgkin's lymphoma (Immunomedics); anti-CD33 mAb conjugate with calicheamicin (CMA 676), gemtuzumab ozogamicin, Mylotarg™, acute myelogenous leukemia (Wyeth); Monopharm-C, colon, lung and pancreatic cancer (Viventia Biotech); anti-idiotypic human mAb to GD2 ganglioside, 4B5, melanoma, small-cell lung cancer, neuroblastoma (Viventia Biotech); anti-EGF receptor humanized Ab, ior egf/r3, cancers of epithelial origin (Center of Molecular Immunology); anti-ior c2 glycoprotein mAb, ior c5, colorectal and ovarian cancer (Center of Molecular Immunology); BABS (biosynthetic antibody binding site) proteins, breast cancer (Chiron); anti-FLK-2/FLT-3 mAb, cancer (tumor-associated angiogenesis) (ImClone Systems); mAb/small-molecule conjugate, TAP (tumor-activated prodrug), cancer (ImmunoGen); anti-GD-2 bispecific mAb, MDX-260, melanoma, glioma, neuroblastoma (Medarex); antinuclear autoantibodies (binds nucleosomes), ANA Ab, cancer (Procyon Biopharma); anti-HLA-DR Ab (SMART 1D10 Ab), Remitogen™, Non-Hodgkin's B-cell lymphoma (Protein Design Labs); SMART ABL 364 Ab, epithelial cell cancers, breast, lung and colon cancer (Protein Design Labs/Novartis); anti-CEA I131-labeled mAb, ImmuRAIT-CEA, colorectal cancer (Immunomedics).

Other antibodies that can be used according to the invention include anti-TNFα antibody such as infliximab (Remicade) and etanercept (Enbrel) for rheumatoid arthritis and Crohn's disease palivizumab; anti-RSV antibody for pediatric subjects; bevacizumab, breast cancer; alemtuzumab, Campath-1H, breast and renal cancer, melanoma, B cell chronic lymphocytic leukemia (Millennium and ILEX); BLyS-mAb, fSLE and rheumatoid arthritis; anti-VEGF2, melanoma, breast cancer; anti-Trail receptor; B3 mAb, breast cancer; m 170 mAb, breast cancer; mAB BR96, breast cancer; Abx-Cbl mAb, graft versus host disease.

The invention embraces a number of classes of antibodies and fragments thereof including but not limited to antibodies directed to cancer antigens (as described above), cell surface molecule, stromal cell molecules, extracellular matrix molecules, and tumor vasculature associated molecules.

A cell surface molecule is a molecule that is expressed at the surface of a cell. In addition to an extracellular domain, it may further comprise a transmembrane domain and a cytoplasinic domain. Examples include HER 2, CD20, CD33, EGF receptor, HLA markers such as HLA-DR, CD52, CD1, CEA, CD22, GD2 ganglioside, FLK2/FLT3, VEGF, VEGFR, and the like.

A stromal cell molecule is a molecule expressed by a stromal cell. Examples include but are not limited to FAP and CD26.

An extracellular matrix molecule is a molecule found in the extracellular matrix. Examples include but are not limited to collagen, glycosaminoglycans (GAGs), proteoglycans, elastin, fibronectin and laminin.

A tumor vasculature associated molecule is a molecule expressed by vasculature of a tumor (i.e., a solid cancer rather than a systemic cancer such as leukemia). As with a cancer antigen, a tumor vasculature associated molecule may be expressed by normal vasculature however its presence on vasculature of a tumor makes it a suitable target for anti-cancer therapy. In some instances, the tumor vasculature associated molecule is expressed at a higher level in tumor vasculature than it is in normal vasculature. Examples include but are not limited to endoglin (see U.S. Pat. No. 5,660,827), ELAM-1, VCAM-1, ICAM-1, ligand reactive with LAM-1, MHC class 11 antigens, aminophospholipids such as phosphatidylserine and phosphatidylethanolamine (as described in U.S. Pat. No. 6,312,694), VEGFR1 (FIt-1) and VEGFR2 (KDR/Flk-1), and other tumor vasculature associated antigens such as those described in U.S. Pat. No. 5,776,427. Antibodies to endoglin are described in U.S. Pat. No. 5,660,827 and include TEC-4 and TEC-11, and antibodies that recognize identical epitopes to these antibodies. Antibodies to aminophospholipids are described in U.S. Pat. No. 6,312,694. Antibodies that inhibit VEGF are described in U.S. Pat. No. 6,342,219 and include 2C3 (ATCC PTA 1595). Other antibodies that are specific for tumor vasculature include antibodies that react to a complex of a growth factor and its receptor such as a complex of FGF and the FGFR or a complex of TGFβ and the TGFβR. Antibodies of this latter class are described in U.S. Pat. No. 5,965,132, and include GV39 and GV97.

It is to be understood that the antibodies embraced by the invention include those recited explicitly herein and also those that bind to the same epitope as those recited herein.

Also useful in the invention are antibodies such as the following, all of which are commercially available:

Apoptosis Antibodies:

BAX Antibodies: Anti-Human Bax Antibodies (Monoclonal), Anti-Human Bax Antibodies (Polyclonal), Anti-Murine Bax Antibodies (Monoclonal), Anti-Murine Bax Antibodies (Polyclonal);

Fas/Fas Ligand Antibodies: Anti-Human Fas/Fas Ligand Antibodies, Anti-Murine Fas/Fas Ligand Antibodies Granzyme Antibodies Granzyme B Antibodies;

BCL Antibodies: Anti Cytochrome C Antibodies, Anti-Human BCL Antibodies (Monoclonal), Anti-Human bcl Antibodies (Polyclonal), Anti-Murine bcl Antibodies (Monoclonal), Anti-Murine bcl Antibodies (Polyclonal);

Miscellaneous Apoptosis Antibodies: Anti TRADD, TRAIL, TRAFF, DR3 Antibodies Anti-Human Fas/Fas Ligand Antibodies Anti-Murine Fas/Fas Ligand Antibodies;

Miscellaneous Apoptosis Related Antibodies: BIM Antibodies: Anti Human, Murine bim Antibodies (Polyclonal), Anti-Human, Murine bim Antibodies (Monoclonal);

PARP Antibodies: Anti-Human PARP Antibodies (Monoclonal), Anti-Human PARP Antibodies (Polyclonal) Anti-Murine PARP Antibodies;

Caspase Antibodies: Anti-Human Caspase Antibodies (Monoclonal), Anti-Murine Caspase Antibodies;

Anti-CD Antibodies: Anti-CD29, PL18-5 PanVera, Anti-CD29, PL4-3 PanVera, Anti-CD41a, PT25-2 PanVera, Anti-CD42b, PL52-4 PanVera, Anti-CD42b, GUR20-5 PanVera, Anti-CD42b, WGA-3 PanVeraAnti-CD43, 1D4 PanVera, Anti-CD46, MCP75-6 PanVera, Anti-CD61, PL 11-7 PanVera, Anti-CD61, PL8-5 PanVera, Anti-CD62/P-slctn, PL7-6 PanVera, Anti-CD62/P-slctn, WGA-1 PanVera, Anti-CD154, SF3 PanVera; and anti-CD1, anti-CD2, anti-CD3, anti-CD4, anti-CD5, anti-CD6, anti-CD7, anti-CD8, anti-CD9, anti-CD 10, anti-CD 11, anti-CD12, anti-CD 13, anti-CD14, anti-CD15, anti-CD16, anti-CD17, anti-CD18, anti-CD19, anti-CD20, anti-CD21, anti-CD22, anti-CD23, anti-CD24, anti-CD25, anti-CD26, anti-CD27, anti-CD28, anti-CD29, anti-CD30, anti-CD3 1, anti-CD32, anti-CD33, anti-CD34, anti-CD35, anti-CD36, anti-CD37, anti-CD38, anti-CD39, anti-CD40 anti-CD41, anti-CD42, anti-CD43, anti-CD44, anti-CD45, anti-CD46, anti-CD47, anti-CD48, anti-CD49, anti-CD50, anti-CD51, anti-CD52, anti-CD53, anti-CD54, anti-CD55, anti-CD56, anti-CD57, anti-CD58, anti-CD59, anti-CD60, anti-CD61, anti-CD62, anti-CD63, anti-CD64, anti-CD65, anti-CD66, anti-CD67, anti-CD68, anti-CD69, anti-CD70, anti-CD71, anti-CD72, anti-CD73, anti-CD74, anti-CD75, anti-CD76, anti-CD77, anti-CD78, anti-CD79, anti-CD80, anti-CD81, anti-CD82, anti-CD83, anti-CD84, anti-CD85, anti-CD86, anti-CD87, anti-CD88, anti-CD89, anti-CD90, anti-CD91, anti-CD92, anti-CD93, anti-CD94, anti-CD95, anti-CD96, anti-CD97, anti-CD98, anti-CD99, anti-CD100, anti-CD101, anti-CD102, anti-CD103, anti-CD104, anti-CD105, anti-CD106, anti-CD107, anti-CD108, anti-CD109, anti-CD110, anti-CD111, anti-CD112, anti-CD113, anti-CD114, anti-CD115, anti-CD116, anti-CD117, anti-CD118, anti-CD119, anti-CD120, anti-CD121, anti-CD122, anti-CD123, anti-CD124, anti-CD125, anti-CD126, anti-CD127, anti-CD128, anti-CD129, anti-CD130, anti-CD131, anti-CD132, anti-CD133, anti-CD134, anti-CD135, anti-CD136, anti-CD137, anti-CD138, anti-CD139, anti-CD140, anti-CD141, anti-CD142, anti-CD143, anti-CD144, anti-CD145, anti-CD146, anti-CD147, anti-CD148, anti-CD149, anti-CD150, anti-CD151, anti-CD152, anti-CD153, anti-CD154, anti-CD155, anti-CD156, anti-CD157, anti-CD158, anti-CD159, anti-CD160, anti-CD161, anti-CD162, anti-CD163, anti-CD164, anti-CD165, anti-CD166, anti-CD167, anti-CD168, anti-CD169, anti-CD170, anti-CD171, anti-CD172, anti-CD173, anti-CD174, anti-CD175, anti-CD176, anti-CD177, anti-CD178, anti-CD179, anti-CD180, anti-CD181, anti-CD182, anti-CD183, anti-CD184, anti-CD185, anti-CD186, anti-CD187, anti-CD188, anti-CD189, anti-CD190, anti-CD191, anti-CD192, anti-CD193, anti-CD194, anti-CD195, anti-CD196, anti-CD197, anti-CD198, anti-CD199, anti-CD200, anti-CD201, anti-CD202, anti-CD203, anti-CD204, anti-CD205, anti-CD206, anti-CD207, anti-CD208, anti-CD209, anti-CD210, anti-CD211, anti-CD212, anti-CD213, anti-CD214, anti-CD215, anti-CD216, anti-CD217, anti-CD218, anti-CD219, anti-CD220, anti-CD221, anti-CD222, anti-CD223, anti-CD224, anti-CD225, anti-CD226, anti-CD227, anti-CD228, anti-CD229, anti-CD230, anti-CD23 1, anti-CD232, anti-CD233, anti-CD234, anti-CD235, anti-CD236, anti-CD237, anti-CD238, anti-CD239, anti-CD240 anti-CD241, anti-CD242, anti-CD243, anti-CD244, anti-CD245, anti-CD246, anti-CD247, anti-CD248, anti-CD249, anti-CD250, and the like.

Human Chemokine Antibodies: Human CNTF Antibodies, Human Eotaxin Antibodies, Human Epitherlial Neutrophil Activating Peptide-78, Human Exodus Antibodies, Human GRO Antibodies, Human HCC-1 Antibodies, Human 1-309 Antibodies, Human IP-10 Antibodies, Human 1-TAC Antibodies, Human LIF Antibodies, Human Liver-Expressed Chemokine Antibodies, Human lymphotoxin Antibodies, Human MCP Antibodies, Human MIP Antibodies, Human Monokine Induced by IFN-gamma Antibodies, Human NAP-2 Antibodies, Human NP-1 Antibodies, Human Platelet Factor-4 Antibodies, Human RANTES Antibodies, Human SDF Antibodies, Human TECK Antibodies;

Murine Chemokine Antibodies: Human B-Cell Attracting Murine Chemokine Antibodies, Chemokine-1 Antibodies, Murine Eotaxin Antibodies, Murine Exodus Antibodies, Murine GCP-2 Antibodies, Murine KC Antibodies, Murine MCP Antibodies, Murine MIP Antibodies, Murine RANTES Antibodies, Rat Chemokine Antibodies, Rat Chemokine Antibodies, Rat CNTF Antibodies, Rat GRO Antibodies, Rat MCP Antibodies, Rat MIP Antibodies, Rat RANTES Antibodies;

Cytokine/Cytokine Receptor Antibodies: Human Biotinylated Cytokine/Cytokine Receptor Antibodies, Human IFN Antibodies, Human IL Antibodies, Human Leptin Antibodies, Human Oncostatin Antibodies, Human TNF Antibodies, Human TNF Receptor Family Antibodies, Murine Biotinylated Cytokine/Cytokine Receptor Antibodies, Murine IFN Antibodies, Murine IL Antibodies, Murine TNF Antibodies, Murine TNF Receptor Antibodies; anti-CCR4 antibody;

Rat Cytokine/Cytokine Receptor Antibodies: Rat Biotinylated Cytokine/Cytokine Receptor Antibodies, Rat IFN Antibodies, Rat IL Antibodies, Rat TNF Antibodies;

ECM Antibodies: Collagen/Procollagen, Laminin, Collagen (Human), Laminin (Human), Procollagen (Human), Vitronectin/Vitronectin Receptor, Vitronectin (Human), Vitronectin Receptor (Human), Fibronectin/Fibronectin Receptor, Fibronectin (Human), Fibronectin Receptor (Human);

Growth Factor Antibodies: Human Growth Factor Antibodies, Murine Growth Factor Antibodies, Porcine Growth Factor Antibodies;

Miscellaneous Antibodies: Baculovirus Antibodies, Cadherin Antibodies, Complement Antibodies, C1q Antibodies, VonWillebrand Factor Antibodies, Cre Antibodies, HIV Antibodies, Influenza Antibodies, Human Leptin Antibodies, Murine Leptin Antibodies, Murine CTLA-4 Antibodies, Human CTLA-4 Antibodies, P450 Antibodies, RNA Polymerase Antibodies;

Neurobio Antibodies: Amyloid Antibodies, GFAP Antibodies, Human NGF Antibodies, Human NT-3 Antibodies, Human NT-4 Antibodies.

Still other antibodies can be used in the invention and these include antibodies listed in references such as the MSRS Catalog of Primary Antibodies, and Linscott's Directory.

In some preferred embodiments of the invention, the antibodies are Avastin (bevacizumab), BEC2 (mitumomab), Bexxar (tositumomab), Campath (alemtuzumab), CeaVac, Herceptin (trastuzumab), IMC-C225 (centuximab), LymphoCide (epratuzumab), MDX-210, Mylotarg (gemtuzumab ozogamicin), Panorex (edrecolomab), Rituxan (rituximab), Theragyn (pemtumomab), Zamyl, and Zevalin (ibritumomab tituxetan). The invention also covers antibody fragments thereof.

In some preferred embodiments, the cancer antigen is VEGF, Anti-idiotypic mAb (GD3 ganglioside mimic), CD20, CD52, Anti-idiotypic mAb (CEA mimic), ERBB2, EGFR, CD22, ERBB2 X CD65 (fcγRI), EpCam, PEM and CD33.

The invention encompasses the use of both antibodies and antibody fragments. The antibodies may be monoclonal or polyclonal, and can be prepared by conventional methodology. They may further be isolated or present in an ascites fluid. Such antibodies can be further manipulated to create chimeric or humanized antibodies as will be discussed in greater detail below.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, l. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of co-specific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions has been replaced by FR regions of human origin. Such antibodies, including fragments of intact antibodies with antigen-binding ability, are often referred to as “chimeric” antibodies. Commercial sources of humanized or chimeric antibodies include GenPharm, Xenotech, AbGenix and CeliGeneSys.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The invention is further based, in part, on the surprising discovery that administration of linear or cyclic Formula I compound and an antibody or fragment thereof such as an anti-cancer antibody or antibody fragment, or an anti-microbial antibody or antibody fragment has unexpected benefit over the administration of either agent alone. In some instances, the effect is additive, and in others it is synergistic.

Thus, in one aspect of the invention, the Formula I compound and the anti-cancer antibody or fragment thereof are administered as a synergistic combination in an effective amount to treat or reduce the risk of developing a cancer. As used herein, the term “synergistic” describes an effect resulting from the combination of at least two agents which is greater than the effect of each of the individual agents when used alone. When used together either or both agents may be used at lower doses than would be used if either agent was administered alone. In these embodiments, either agent or both may be administered in a “sub-therapeutic” dose for each alone, the combination, however, being therapeutic.

Treatment after a disorder has started aims to reduce, ameliorate or altogether eliminate the disorder, and/or its associated symptoms, or prevent it from becoming worse. Treatment of subjects before a disorder has started (i.e., prophylactic treatment) aims to reduce the risk of developing the disorder. As used herein, the term “prevent” refers to the prophylactic treatment of patients who are at risk of developing a disorder (resulting in a decrease in the probability that the subject will develop the disorder), and to the inhibition of further development of an already established disorder.

The antibodies provided herein can be used additionally for delivery of toxic substances to cancer cells. Antibodies are commonly conjugated to toxins such as ricin (e.g., from castor beans), calicheamicin and maytansinoids, to radioactive isotopes such as Iodine-131 and Yttrium-90, to chemotherapeutic agents, or to biological response modifiers. In this way, the toxic substances can be concentrated in the region of the cancer and non-specific toxicity to normal cells can be minimized. In addition to the use of antibodies which are specific for cancer antigens, antibodies which bind to vasculature, such as those which bind to endothelial cells, are also useful in the invention. This is because, generally, solid tumors are dependent upon newly formed blood vessels to survive, and thus most tumors are capable of recruiting and stimulating the growth of new blood vessels. As a result, one strategy of many cancer medicaments is to attack the blood vessels feeding a tumor and/or the connective tissues (or stroma) supporting such blood vessels.

The compositions of the invention can further include chemotherapeutic agents such as but not limited to those currently in use with the antibodies recited herein. Several chemotherapeutic agents can be categorized as DNA damaging agents and these include topoisomerase inhibitors (e.g., etoposide, ramptothecin, topotecan, teniposide, mitoxantrone), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), and radiation therapy.

Important anticancer agents are those selected from the group consisting of: Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N, N′-Bis(2-chloroethyl)-N-nitrosourea (BCNU); N-(2-chloroethyl)-N′-cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); 2-chlorodeoxyadenosine (2-Cda).

Other anti-neoplastic compounds include: 20-epi-1,25 dihydroxyvitamnin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A₂; bleomycin B₂; bropirimine; budotitane; buthionine sulfoximine; calcipotriol; calphostin C; camptothecin derivatives (e.g., 10-hydroxy- camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones (A, R=H; B, R=Me); epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptoistatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix met alloproteinase inhibitors; menogaril; merbarone; meterelin; methioninase; metoclopramide; MIF inhibitor; mifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosanninoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Other anti-cancer agents include: Antiproliferative agents (e.g., Piritrexim Isothionate), Antiprostatic hypertrophy agent (e.g., Sitogluside), Benign prostatic hyperplasia therapy agents (e.g., Tamsulosin Hydrochloride), Prostate growth inhibitor agents (e.g., Pentomone), and Radioactive agents: Fibrinogen 1 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; Triolein I 131.

Another category of anti-cancer agents is anti-cancer Supplementary Potentiating Agents, including: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL.

Particularly important anticancer agents are those selected from the group consisting of: annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO₄; mitoxantrone; mitozolomide; Pentostatin; Tomudex.

One particularly preferred class of anticancer agents are taxanes (e.g., paclitaxel and docetaxel) are preferred. Another important category of anticancer agent is annonaceous acetogenin.

In important embodiments, the agents are administered together with anti-cancer compounds selected from the group consisting of aldesleukin, asparaginase, bleomycin sulfate, carboplatin, chlorambucil, cisplatin, cladribine, cyclophosphamide, cytarabine, dacarbazine, dactinomycin, daunorubicin hydrochloride, docetaxel, doxorubicin, doxorubicin hydrochloride, epirubicin hydrochloride, etoposide, etoposide phosphate, floxuridine, fludarabine, fluorouracil, gemcitabine, gemcitabine hydrochloride, hydroxyurea, idarubicin hydrochloride, ifosfamide, interferons, interferon-α2a, interferon-α2b, interferon-αn3, interferon-α1b, interleukins, irinotecan, mechlorethamine hydrochloride, melphalan, mercatopurine, methotrexate, methotrexate sodium, mitomycin, mitoxantrone, paclitaxel, pegaspargase, pentostatin, prednisone, profimer sodium, procabazine hydrochloride, taxol, taxotere, teniposide, topotecan hydrochloride, vinblastine sulfate, vincristine sulfate and vinorelbine tartrate.

Other cancer therapies include hormonal manipulation, particularly for breast and gynecological cancers. Formula I compounds are also useful in combination with tamoxifen or aromatase inhibitor arimidex (i.e., anastrozole), or simply for disorders responsive to either (e.g., breast cancer).

Formula I compounds can also be combined, and/or administered substantially simultaneously, with enzyme inhibitor agents such as CDK inhibitors, tyrosine kinase inhibitors, MAP kinase inhibitors, and EGFR inhibitors (e.g., C225).

The combination therapy is administered to subjects having or at risk of developing cancer. A subject having a cancer is a subject that has detectable cancerous cells. A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

A cancer cell is a cell that divides and reproduces abnormally due to a loss of normal growth control. Cancer cells almost always arise from at least one genetic mutation. In some instances, it is possible to distinguish cancer cells from their normal counterparts based on profiles of expressed genes and proteins, as well as to the level of their expression. Genes commonly affected in cancer cells include oncogenes, such as ras, neu/HER2/erbB, myb, myc and abl, as well as tumor suppressor genes such as p53, Rb, DCC, RET and WT. Cancer-related mutations in some of these genes leads to a decrease in their expression or a complete deletion. In others, mutations cause an increase in expression or the expression of an activated variant of the normal counterpart.

The term “tumor” is usually equated with neoplasm, which literally means “new growth” and is used interchangeably with “cancer.” A “neoplastic disorder” is any disorder associated with cell proliferation, specifically with a neoplasm. A “neoplasm” is an abnormal mass of tissue that persists and proliferates after withdrawal of the carcinogenic factor that initiated its appearance. There are two types of neoplasms, benign and malignant. Nearly all benign tumors are encapsulated and are noninvasive; in contrast, malignant tumors are almost never encapsulated but invade adjacent tissue by infiltrative destructive growth. This infiltrative growth can be followed by tumor cells implanting at sites discontinuous with the original tumor. The method of the invention can be used to treat neoplastic disorders in humans, including but not limited to: sarcoma, carcinoma, fibroma, leukemia, lymphoma, melanoma, myeloma, neuroblastoma, rhabdomyosarcoma, retinoblastoma, and glioma as well as each of the other tumors described herein.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

Carcinomas are cancers of epithelial origin. Carcinomas intended for treatment with the methods of the invention include, but are not limited to, acinar carcinoma, acinous carcinoma, alveolar adenocarcinoma (also called adenocystic carcinoma, adenomyoepithelioina, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Huirthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypernephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the invention are used to treat subjects having cancer of the breast, cervix, ovary, prostate, lung, colon and rectum, pancreas, stomach or kidney.

Another particularly important cancer type is sarcomas. Sarcomas are rare mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., non-bone) Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

The cancers to be treated may be refractory cancers. A refractory cancer as used herein is a cancer that is resistant to the ordinary standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, or surgery, or radiation, or a combination thereof. Those of ordinary skill in the art are aware of such standards of care. Subjects being treated according to the invention for a refractory cancer therefore may have already been exposed to another treatment for their cancer. Alternatively, if the cancer is likely to be refractory (e.g., given an analysis of the cancer cells or history of the subject), then the subject may not have already been exposed to another treatment.

Examples of refractory cancers include but are not limited to leukemias, melanomas, renal cell carcinomas, colon cancer, liver (hepatic) cancers, pancreatic cancer, Non-Hodgkin's lymphoma, and lung cancer.

The invention can also be used to treat cancers that are immunogenic. Cancers that are immunogenic are cancers that are known to (or likely to) express immunogens on their surface or upon cell death. These immunogens are in vivo endogenous sources of cancer antigens and their release can be exploited by the methods of the invention in order to treat the cancer. Examples of immunogenic cancers include those listed in Table 1, including malignant melanoma and renal cell cancer.

Subjects at risk of developing a cancer include subjects that are known or are suspected of being exposed to a carcinogen. A carcinogen is an agent capable of initiating development of malignant cancers. Exposure to carcinogens generally increases the risk of neoplasms in subjects, usually by affecting DNA directly. Carcinogens may take one of several forms such as chemical, electromagnetic radiation, or may be an inert solid body. Examples of chemical carcinogens include tobacco, asbestos, and the like.

The goal of immunotherapy is to augment a patient's immune response to an established tumor. Different types of cells that can kill tumor targets in vitro and in vivo have been identified: natural killer cells (NK cells), cytolytic T lymphocytes (CTLs), lymphokine-activated killer cells (LAKs), activated macrophages, and neutrophils. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class 1. CTLs are thought to be effector cells in the rejection of transplanted tumors and of tumors caused by DNA viruses. LAK cells are a subset of null lymphocytes distinct from the NK and CTL populations. Activated macrophages and neutrophils can directly kill tumor cells in a manner that is not antigen dependent nor MHC restricted. In addition, neutrophils can inhibit tumor growth by killing endothelial cells of the vasculature that provide blood supply to the tumor. Thus, activated macrophages and neutrophils are thought to decrease the growth rate of the tumors they infiltrate.

The vaccine methods and compositions described herein similarly envision the use of nucleic acid based vaccines in addition to peptide based vaccines. The art is familiar with nucleic acid based vaccines.

The invention seeks to enhance other forms of immunotherapy including dendritic cell vaccines. These vaccines generally include dendritic cells loaded ex vivo with antigens such as tumor-associated antigens. The dendritic cells can be incubated with the antigen, thereby allowing for antigen processing and expression on the cell surface, or the cells may simply be combined with the antigen prior to injection in vivo. Alternatively, the dendritic cells may be activated in vitro and then re-infused into a subject in the activated state. Formula I compounds can be combined with the dendritic cells in all of these embodiments. Examples of dendritic cell based vaccines include autologous tumour antigen-pulsed dendritic cells (advanced gynaecological malignancies); blood-derived dendritic cells loaded ex vivo with prostate cancer antigen (Provenge; Dendreon Corporation); blood-derived dendritic cells loaded ex vivo with antigen for multiple myeloma and other B-cell malignancies (Mylovenge; Dendreon Corporation); and blood-derived dendritic cells loaded ex vivo with antigen for cancers expressing the HER-2/neu proto-oncogene (APC8024; Dendreon Corporation); xenoantigen (e.g., PAP) loaded dendritic cells, and the like.

One advantage of the combined use of Formula I compounds and the foregoing vaccines is the reduction in the number of immunizations that a subject must receive in order to achieve a therapeutically or prophylactically effective immune response. For example, for some infectious diseases, three or more vaccinations are required before a fully effective immune response is generated and the subject is immunized. This number can be reduced by combining Formula I compound administration with the vaccine, either physically or temporally. Accordingly, Formula I compounds are particularly suited to subjects at risk of infectious disease.

Another form of immunotherapy is the use of lymphokine activated killer cells (LAKs) that are primed in vitro with lymphokines and then re-infused into a subject. The agents of Formula I can be combined with such cells either as an addition to the activating lymphokine or in place of it.

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops). Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. Although many of the embodiments described herein relate to human disorders, the invention is also useful for treating other nonhuman vertebrates.

The invention also embraces the use of adjuvants. Adjuvant substances derived from microorganisms, such as bacillus Calmette-Guerin, heighten the immune response and enhance resistance to tumors in animals. Adjuvants that may be combined with the compounds of Formula I include alum, immunostimulatory oligonucleotides such as CpG oligonucleotides, QS-21, and the like. These and other adjuvants are listed herein in greater detail.

The term “effective amount” of either or the combination of compounds refers to the amount necessary or sufficient to realize a desired biologic effect. For example, an effective amount of the combination could be that amount necessary to cause activation of the immune system, resulting potentially in the development of an antigen specific immune response. Generally, an effective amount is that amount that provides a biologically beneficial effect. The biologically beneficial effect may be the amelioration and or absolute elimination of symptoms resulting from the disorder being treated e.g., cancer or infectious disease. In another embodiment, the biologically beneficial effect is the complete abrogation of the disorder e.g., cancer, as evidenced for example, by the absence of a tumor or a biopsy or blood smear which is free of cancer cells.

The effective amount may vary depending upon the particular compound and the particular antibody used. The effective amount for any particular application can also vary depending on such factors as the cancer being treated, the size of the subject, or the severity of the disease or condition. One of ordinary skill in the art can empirically determine the effective amount of a particular Formula I compound and anti-cancer antibody combination without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

In some instances, a sub-therapeutic dosage of either the Formula I compound or the anti-cancer treatment, or a sub-therapeutic dosage of both, is used in the treatment of a subject having, or at risk of developing, cancer. As an example, it has been discovered according to the invention, that when the two classes of drugs are used together, the anti-cancer antibody can be administered in a sub-therapeutic dose and still produce a desirable therapeutic result. A “sub-therapeutic dose” as used herein refers to a dosage which is less than that dosage which would produce a therapeutic result in the subject if administered in the absence of the other agent. Thus, the sub-therapeutic dose of a anti-cancer antibody is one which would not produce the desired therapeutic result in the subject in the absence of the administration of the Formula I compound. Therapeutic doses of anti-cancer antibodies are well known in the field of medicine for the treatment of cancer. These dosages have been extensively described in references such as Remington's Pharmaceutical Sciences, 18th ed., 1990, or the Physician Desktop Reference; as well as many other medical references relied upon by the medical profession as guidance for the treatment of cancer.

For any compound described herein a therapeutically effective amount can be initially determined from cell culture assays. In particular, the effective amount of a Formula I compound can be determined using in vitro stimulation assays. The stimulation index of immune cells can be used to determine an effective amount of the particular compound for the particular subject, and the dosage can be adjusted upwards or downwards to achieve the desired levels in the subject.

Therapeutically effective amounts can also be determined in animal studies. For instance, the effective amount of a Formula I compound and an anti-cancer antibody to induce a synergistic response can be assessed using in vivo assays of tumor regression and/or prevention of tumor formation. Relevant animal models include assays in which malignant cells are injected into the animal subjects, usually in a defined site. Generally, a range of Formula I compound doses are administered into the animal along with a range of anti-cancer antibody doses. Inhibition of the growth of a tumor following the injection of the malignant cells is indicative of the ability to reduce the risk of developing a cancer. Inhibition of further growth (or reduction in size) of a pre-existing tumor is indicative of the ability to treat the cancer. Mice which have been modified to have human immune system elements can be used as recipients of human cancer cell lines to determine the effective amount of the synergistic combination.

The applied dose of both agents can be adjusted based on the relative bioavailability and potency of the administered compounds, including the adjuvants used. Adjusting the dose to achieve maximal efficacy based on the methods described above and other methods are well within the capabilities of the ordinarily skilled artisan.

Subject doses of the compounds described herein typically range from about 0.1 μg to 10,000 mg, more typically from about 1 μg/day to 8000 mg, even more typically from about 10 μg to 5 mg, and most typically from about 10 μg to 100 pg. Stated in terms of subject body weight, typical dosages range from about 0.1 μg to 20 mg/kg/day, more typically from about 1 to 10 mg/kg/day, and most typically from about 1 to 5 mg/kg/day.

In particularly important embodiments, the agent is administered in amounts of less than or equal to 1.0 mg/kg per day. This includes amounts equal to or less than 0.9, 0.8, 0.7, 0.6, 0.5, 0.4, 0.3, 0.2, 0.1 mg/kg per day. The agents may also be administered in amounts of less than or equal to 0.1 mg/kg per day (e.g., less than or equal to 0.09, 0.08, 0.07, 0.06, 0.5, 0.04, 0.03, 0.02 or 0.01 mg/kg/day). In some embodiments, the agents are administered in a range of about 0.005 mg/kg per day to less than 1.0 mg/kg per day (or about 0.005 mg/kg per day to equal to or less than 0.1 mg/kg per day).

In methods particularly directed at subjects at risk of developing a disorder, timing of the administration of the agent of Formula I and the anti-cancer antibody or antibody fragment may be particularly important. For instance, in a subject with a genetic predisposition to cancer, the agents may be administered to the subject on a routine schedule.

A “routine schedule” as used herein, refers to a predetermined designated period of time. The routine schedule may encompass periods of time which are identical or which differ in length, as long as the schedule is predetermined. For instance, the routine schedule may involve administration on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration on a daily basis for the first week, followed by a monthly basis for several months, and then every three months after that. Any particular combination would be covered by the routine schedule as long as it is determined ahead of time that the appropriate schedule involves administration on a certain day.

The compounds of the invention may be administered neat, or in the context of a vector or delivery system. An example of a chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vessels (LUV), which range in size from 0.2-4.0 μm can encapsulate large macromolecules. RNA, DNA and intact virions can be encapsulated within the aqueous interior and be delivered to cells in a biologically active form (Fraley, et al., Trends Biochem. Sci., (1981) 6:77).

Liposomes may be targeted to a particular tissue by coupling the liposome to a specific ligand such as a sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammoniuin bromide (DDAB). Methods for making liposomes are well known in the art and have been described in many publications. Liposomes also have been reviewed by Gregoriadis, G. in Trends in Biotechnology, (1985) 3:235-241.

In another embodiment the chemical/physical vector is a biocompatible microsphere that is suitable for delivery, such as oral or mucosal delivery. Such microspheres are disclosed in Chickering et al., Biotech. And Bioeng., (1996) 52:96-101 and Mathiowitz et al., Nature, (1997) 386:.410-414 and PCT Patent Application WO97/03702.

Both non-biodegradable and biodegradable polymeric matrices can be used to deliver the Formula I compound and/or the anti-cancer antibody to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

The polymeric matrix preferably is in the form of a microparticle such as a microsphere (wherein the agents are dispersed throughout a solid polymeric matrix) or a microcapsule (wherein the agents are stored in the core of a polymeric shell). Other forms of the polymeric matrix for containing the agents include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the Formula I compound and the anti-cancer antibody are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the Formula I compounds are administered to the subject via an implant while the anti-cancer antibody is administered acutely.

Bioadhesive polymers of particular interest include bioerodible hydrogels described by H. S. Sawhney, C. P. Pathak and J. A. Hubell in Macromolecules, (1993) 26:581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(laurel methacrylate), poly(phenyl methacrylate), poly(methyl acrylate), poly(isopropyl acrylate), poly(isobutyl acrylate), and poly(octadecyl acrylate).

Other delivery vehicles can be used and these include: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The compositions and methods of the invention in certain instances may be useful for replacing existing surgical procedures or drug therapies, although in most instances the present invention is useful in improving the efficacy of existing therapies for treating such conditions. Accordingly combination therapy may be used to treat the subjects that are undergoing or that will undergo a treatment for inter alia cancer or infectious disease. For example, the agents may be administered to a subject in combination with another anti-proliferative (e.g., an anti-cancer) therapy. Suitable anti-cancer therapies include surgical procedures to remove the tumor mass, chemotherapy or localized radiation. The other anti-proliferative therapy may be administered before, concurrent with, or after treatment with the agent of the invention. There may also be a delay of several hours, days and in some instances weeks between the administration of the different treatments, such that the agent may be administered before or after the other treatment. In some embodiments, the agents of Formula I may be administered with or without the antigens or antibodies, prior to the administration of the other anti-proliferative treatment (e.g., prior to surgery, radiation or chemotherapy), although the timing is not so limited. Although not intending to be bound by any particular mechanism, it is proposed that the administration of Formula I compounds inducing memory within the immune cell compartment, for example, by the induction of memory T cells, and B cells. This is believed to occur via the cytokine cocktail that is induced by compounds of Formula I, particularly the induction of IL-1. The ability to generate memory T cells can enhance immune responses to, for example, cancerous cells that are remaining following a surgical procedure, or following chemotherapy or radiation. The invention further contemplates the use of Formula I compounds in cancer subjects prior to and following surgery, radiation or chemotherapy in order to create memory immune cells to the cancer antigen. In this way, memory cells of the immune system can be primed with cancer antigens and thereby provide immune surveillance in the long term. This is particularly suited to radiotherapy of subjects where immune cells so primed can invade a tumor site and effectively clear any remaining tumor debris. This in turn promotes further immunity to the cancer, particularly to antigens that might not have been exposed in the context of a tumor mass pre-treatment.

It is to be understood that in other embodiments, the subjects can be treated with Formula I compounds without any other therapy, as well. In some important embodiments of the invention, the methods are particularly directed to subjects at high risk of cancer, such as those predisposed for familial (e.g., familial colon polyposis, BRCA1- or BRCA2-associated breast cancer, Wilms tumour, colorectal cancer, Li-Fraumeni Syndrome, ovarian cancer, and prostate cancer), or non-familial genetic reasons. Subjects at high risk are also those that manifest pre-cancerous symptoms such as pre-cancerous polyps (e.g., in colon cancer), or pre-cancerous lesions (e.g., in HPV-induced cervical cancer).

The agents can also be administered in combination with non-surgical anti-proliferative (e.g., anti-cancer) drug therapy. In one embodiment, the agent may be administered in combination with an anti-cancer compound such as a cytostatic compound. A cytostatic compound is a compound (e.g., a nucleic acid, a protein) that suppresses cell growth and/or proliferation. In some embodiments, the cytostatic compound is directed towards the malignant cells of a tumor. In yet other embodiments, the cytostatic compound is one which inhibits the growth and/or proliferation of vascular smooth muscle cells or fibroblasts.

According to the methods of the invention, Formula I compounds and the anti-cancer antibodies may be administered prior to, concurrent with, or following other anti-cancer compounds. The administration schedule may involve administering the different agents in an alternating fashion. In other embodiments, the combination therapy of the invention may be delivered before and during, or during and after, or before and after treatment with other therapies. In some cases, the agent is administered more than 24 hours before the administration of the other anti-proliferative treatment. In other embodiments, more than one anti-proliferative therapy may be administered to a subject. For example, the subject may receive the agents of the invention, in combination with both surgery and at least one other anti-proliferative compound. Alternatively, the agent may be administered in combination with more than one anti-cancer drug.

The Formula I compounds and anti-cancer antibodies can be combined with other therapeutic agents such as adjuvants to enhance immune responses even further. The Formula I compound, anti-cancer antibody, and other therapeutic agent may be administered simultaneously or sequentially. When the other therapeutic agents are administered simultaneously they can be administered in the same or separate formulations, but are administered at the same time. The administration of the other therapeutic agents (such as adjuvants) and the Formula I compounds and anti-cancer antibodies can also be temporally separated, meaning that the therapeutic agents are administered at a different time, either before or after, the administration of the Formula I compounds and anti-cancer antibodies. The separation in time between the administration of these compounds may be a matter of minutes or it may be longer. Other therapeutic agents include but are not limited to nucleic acid adjuvants, non-nucleic acid adjuvants, cytokines, non-immunotherapeutic antibodies, antigens, etc.

A nucleic acid adjuvant is an adjuvant that is a nucleic acid. Examples include immunostimulatory nucleic acid molecules such as those containing CpG dinucleotides, as described in U.S. Pat. No. 6,194,388B1, issued Feb. 27, 2001, U.S. Pat. No. 6,207,646 B1, issued Mar. 27, 2001, and U.S. Pat. No. 6,239,116 B1, issued May 29, 2001.

A “non-nucleic acid adjuvant” is any molecule or compound except for the immunostimulatory nucleic acids described herein which can stimulate the humoral and/or cellular immune response. Non-nucleic acid adjuvants include, for instance, adjuvants that create a depo effect, immune-stimulating adjuvants, adjuvants that create a depo effect and stimulate the immune system and mucosal adjuvants.

An “adjuvant that creates a depo effect” as used herein is an adjuvant that causes an antigen, such as a cancer antigen present in a cancer vaccine, to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC Pharmaceuticals Corporation, San Diego, Calif.).

An “immune stimulating adjuvant” is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Antigenics, Inc., Waltham, Mass.); poly [di (carboxylatophenoxy) phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.).

“Adjuvants that create a depo effect and stimulate the immune system” are those compounds which have both of the above-identified functions. This class of adjuvants includes but is not limited to ISCOMS (Immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A “non-nucleic acid mucosal adjuvant” as used herein is an adjuvant other than an immunostimulatory nucleic acid that is capable of inducing a mucosal immune response in a subject when administered to a mucosal surface in conjunction with an antigen. Mucosal adjuvants include but are not limited to Bacterial toxins: e.g., Cholera toxin (CT), CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995), Zonula occludens toxin, zot, Escherichia coli heat-labile enterotoxin, Labile Toxin (LT), LT derivatives including but not limited to LT B subunit (LTB) (Verweij et al., 1998); LT7K (Arg to Lys) (Komase et al., 1998, Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998, Douce et al., 1997, 1998, Di Tommaso et al., 1996); and LTR72 (Ala to Arg) (Giuliani et al., 1998), Pertussis toxin, PT. (Lycke et al., 1992, Spangler B D, 1992, Freytag and Clemments, 1999, Roberts et al., 1995, Wilson et al., 1995) including PT-9K/129G (Roberts et al., 1995, Cropley et al., 1995); Toxin derivatives (see below) (Holmgren et al., 1993, Verweij et al., 1998, Rappuoli et al., 1995, Freytag and Clements, 1999); Lipid A derivatives (e.g., monophosphoryl lipid A, MPL) (Sasaki et al., 1998, Vancott et al., 1998; Muramyl Dipeptide (MDP) derivatives (Fukushima et al., 1996, Ogawa et al., 1989, Michalek et al., 1983, Morisaki et al., 1983); Bacterial outer membrane proteins (e.g., outer surface protein A (OspA) lipoprotein of Borrelia hurgdorferi, outer membrane protine of Neisseria meningitidis)(Marinaro et al., 1999, Van de Verg et al., 1996); Oil-in-water emulsions (e.g., MF59) (Barchfield et al., 1999, Verschoor et al., 1999, O'Hagan, 1998); Aluminum salts (Isaka et al., 1998, 1999); and Saponins (e.g., QS21) Aquila Biopharmaceuticals, Inc., Worcester, Mass.) (Sasaki et al., 1998, MacNeal et al., 1998), ISCOMS, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micell-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA) and Leishmania elongation factor (Corixa Corporation, Seattle, Wash.).

Cytokines and chemokines can potentially be cleaved and thereby inactivated by post proline cleaving enzymes. Administration of Formula I compounds with cytokines and/or chemokines can enhance the efficacy of these latter agents by protecting them from degradation.

Immune responses can also be induced or augmented by the co-administration or co-linear expression of cytokines or chemokines (Bueler & Mulligan, 1996; Chow et al., 1997; Geissler et al., 1997; Iwasaki et al., 1997; Kim et al., 1997) or B-7 co-stimulatory molecules (Iwasaki et al., 1997; Tsuji et al., 1997) with the Formula I compounds and anti-cancer antibodies. The cytokines and/or chemokines can be administered directly or may be administered in the form of a nucleic acid vector that encodes the cytokine, such that the cytokine can be expressed in vivo. In one embodiment, the cytokine or chemokine is administered in the form of a plasmid expression vector. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Cytokines also are central in directing the T cell response. Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (G-CSF), interferon-γ (IFN-γ), IFN-α, tumor necrosis factor (TNF), TGF-β, FLT-3 ligand, and CD40 ligand. In some embodiments, the cytokine is a Th1 cytokine. In still other embodiments, the cytokine is a Th2 cytokine.

The term “chemokine” is used as a generic name for peptides or polypeptides that act principally to chemoattract effector cells of both innate and adaptive immunity. Chemokines are thought to coordinate immunological defenses against tumors and infectious agents by concentrating neutrophils, macrophages, eosinophils and T and B lymphocytes at the anatomical site in which the tumor or infectious agent is present. In addition, many chemokines are known to activate the effector cells so that their immune functions (e.g., cytolysis of tumor cells) are enhanced on a per cell basis. Two groups of chemokines are distinguished according to the positions of the first two cysteine residues that are conserved in the amino-terminal portions of the polypeptides. The residues can either be adjacent or separated by one amino acid, thereby defining the CC and CXC cytokines respectively. The activity of each chemokine is restricted to particular effector cells, and this specificity results from a cognate interaction between the chemokine and a specific cell membrane receptor expressed by the effector cells. For example, the CXC chemokines IL-8, Groα/β and ENA 78 act specifically on neutrophils, whereas the CC cheinokines RANTES, MIP-1α and MCP-3 act oil monocytes and activated T cells. In addition, the CXC chemokine IP-10 appears to have anti-angiogenic activity against tumors as well as being a chemoattractant for activated T cells. MIP-1α also reportedly has effects on hemopoietic precursor cells.

In other aspects, the invention relates to kits that are useful in the treatment of cancer. One kit of the invention includes a sustained release vehicle containing a Formula I compound and a container housing an anti-cancer antibody (or an antigen) and instructions for timing of administration of the both. A sustained release vehicle is used herein in accordance with its prior art meaning of any device which slowly releases the Formula I compound.

Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The pharmaceutical compositions of the invention contain an effective amount of a Formula I compound and anti-cancer antibody and/or an antigen and/or other therapeutic agents, optionally included in a pharmaceutically-acceptable carrier. The term “pharmaceutically-acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The agents may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline met al or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Another suitable compound for sustained release delivery is GELFOAM, a commercially available product consisting of modified collagen fibers.

Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The agents can be administered by any ordinary route for administering medications. Depending upon the type of cancer to be treated, the Formula I compounds and anti-cancer antibodies of the invention may be inhaled, ingested or administered by systemic routes. Systemic routes include oral and parenteral. Inhaled medications are preferred in some embodiments because of the direct delivery to the lung, particularly in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For use in therapy, an effective amount of the Formula I compound can be administered to a subject by any mode that delivers the compound to the affected organ or tissue, or alternatively to the immune system. “Administering” the pharmaceutical composition of the present invention may be accomplished by any means known to the skilled artisan. Preferred routes of administration include but are not limited to oral, parenteral, intramuscular, intranasal, intratracheal, inhalation, ocular, vaginal, and rectal.

The administration route of the Formula I compound and the other agents described herein is not limiting on the administration route of the antibody, antibody fragment or antigen described herein. The Formula I compound may be administered in the same route, and in the same formulation as the antibody, antibody fragment or antigen, or it may be administered in a different route, different formulation, and even on a different schedule. In an important embodiment, the Formula I compound is administered orally, and the antibody, antibody fragment or antigen is administered parenterally, preferably by intramuscular or subcutaneous injection, although it is not so limited.

In some important embodiments, the antigens or antibodies are administered mucosally. In these and other embodiments, the subject may be passively or actively exposed to an antigen. Passive exposure occurs when the subject comes in contact with an antigen, such as an infectious pathogen, by being in an environment in which the pathogen is present, and unbeknownst to the subject. Active exposure on the other hand occurs when the subject is deliberately administered an antigen generally for the purpose of vaccination. Passive exposure to infectious pathogens often occurs at the mucosal surfaces such as the oral, nasal, vaginal, penile, and rectal surfaces. Accordingly, the invention embraces exposure of antigens at these surfaces, prior to, substantially simultaneously with, and/or following administration of compounds of Formula I.

In some embodiments, it is preferred that antigens and antibodies by administered by routes that mimic the routes through which antigens or carcinogens would enter the body of the subject. For example, if the antigen is from a respiratory virus, then in some instances it is preferable to administer the antigen by inhalation. Similarly, if the antigen is from a microbe that is generally transmitted by sexual intercourse, then in some instances it is preferable to administer such antigens or antibodies to a vaginal, penile or rectal surface.

In some important embodiments, the compounds of Formula I are administered orally, preferably by ingestible tablets that enter the gastrointestinal tract. In some embodiments, the antigens or antibodies are also administered via the same route. In some instances, it is preferred that the Formula I compounds be formulated together with the antigens, and this may be the case particularly in subjects that have or are at risk of developing an HIIV infection.

In still other embodiments, the Formula I compounds are administered locally, and optionally the antigens or antibodies are administered locally as well.

For oral administration, the agents can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

In some important embodiments, the timing of administration of the Formula I compound and the antigen are important. Thus, the invention embraces the administration of a Formula I compound, preferably with an antigen, prior to treatment with other conventional therapy. For example, if the subject has cancer, then conventional therapy includes surgical removal of a tumor, radiation therapy, or chemotherapy. It is preferred in some instances to administer the Formula I compound with antigen prior to this therapy, and even more preferred to administer the Formula I compound with antigen after this therapy as well. Thus, the method would involve both a prime and a boost dose to antigen (with the Formula I compound). In some embodiments, the antigen alone can be administered particularly in the boost dose.

In embodiments involving the administration of Formula 1 μgents and an antibody such as the anti-HER2 antibody trastuzumab (Herceptin™), the antibody may be administered initially in a dose of 4 mg/kg (dose/unit body weight) as a 90 minute infusion followed by a weekly maintenance dose of 2 mg/kg. In embodiments involving the administration of Formula 1 μgents and an antibody such as the anti-CD20 antibody rituximab (Rituxan™), the antibody may be administered in weekly infusions for 4 or 8 doses (i.e., for 4-8 weeks), each dose being 375 mg/mn² (dose/unit body surface area). Formula I compounds could be administered, twice daily, for a period immediately prior to the initial antibody dose (e.g., 7 days). Since Formula I compounds will expand immune effector cells (e.g., neutrophils, macrophages, eosinophils and T lymphocytes) and direct them to the microenvironment of the tumor, pretreatment with such compounds will accelerate cytotoxicity mediated by the subsequent administration of antibody. Thus, Formula I compounds can be used solely in a pretreatment regime (i.e., prior to exposure to the antibody), or in a combination of pre- and post-treatment administrations. As a non-limiting example of this latter embodiment, pre-treatment with a Formula I compound can be followed by subsequent courses of defined period (e.g., 7 days) administration that could either be concurrent or spaced by intervals (e.g., 7 day pretreatment, 7 day gap, 7 day treatment etc.). Antibody treatment would be continue weekly as currently recommended by the manufacturer (e.g., Genentech, Inc., IDEC Pharmaceuticals, etc.).

The antibody or antibody fragment may be administered together with the agent of Formula I in a multi-day cycle. The multi-day cycle be a 2, 3, 4, 5, 6, 7, 8, 9, 10, or more day cycle. The antibody or fragment thereof may be administered on the first day of such a cycle, followed by administration of the Formula I Agent for a number of days, which may or may not be consecutive. For example, the Formula I Agent may be administered on all remaining days of a multi-day cycle. The Formula I Agent may be administered once, twice, thrice, or more times per day as well. The multi-day cycle may be repeated once, twice, thrice, or more times. Alternatively, it may be repeated for a length of time such as a week, a month, two months, or more, depending upon the status of the subject and the therapeutic response observed. As an non-limiting example, the antibody or fragment thereof is administered on the first day of a seven day cycle, and the Formula I Agent is administered twice a day for the remaining six days of the seven day cycle. The seven day cycle is performed four times resulting in a 28 day treatment.

The invention further provides kits that minimally comprise the agents of the invention. As an example, the kits may comprise in one container the antibody or antibody fragment, preferably formulated and contained for administration by injection, and in another container the compound of Formula I, preferably formulated for oral administration (e.g., as a tablet). As another example, the kits may comprise in one container both the compound of Formula I and an antigen, or a cocktail of antigens. Alternatively, the Formula I compounds and the antigens may be provided in the same kit but in different containers, and in different formulations for different administration routes. In some embodiments, it is preferred to provide all the active agents in a powdered form such as a lyophilized form that can be reconstituted prior to administration to a subject. All the kits of the invention can optionally contain instructions for storage, reconstitution (if applicable) and administration.

### EXAMPLES

### Example 1

### PT-100 Increases Cytokine and Chemokine Gene Expression Early During Treatment of WEHI 164 Tumors

Mice were inoculated with WEHI 164 cells, and starting 2 days later, administered (twice daily) a 5 μg dose of PT-100 or saline (control). RNA extracted from lymph nodes and tumors 2 hours after the first dose of PT-100 on day 4 after tumor inoculation was processed, labeled and hybridized to Affymetrix GeneChips according to the manufacturer's instructions. The log ratio of expression values (PT-100 treated:saline treatment) has been plotted on the ordinates for the cytokine and chemokine genes indicated on the abscissae. Zero values indicate that gene expression was either undetectable or unaffected by PT-100 treatment. The data show selective induction of cytokine and chemokine gene expression in both the tumor and the draining inguinal lymph nodes.

### Example 2

### Function of Cytokines and Chemokines Induced by PT-100

Table 2 lists the effector cell types involved in innate and specific T cell-mediated immunity that are affected by the cytokines and chemokines up-regulated by PT-100 in tumors and draining lymph nodes as described in Example 1. IL-1α and IL-1β, G-CSF, IL-6, and IFN-β either act alone or in combination with other cytokines to stimulate proliferation and/or activation of the indicated effector cell types. MCP-2, MARC/MCP-3, MCP-5, JE, IL-8 (or KC in mice), ENA78, LIX, Lymphotactin, MIG, IP-10, MDC, and TARC are chemokines that act to chemo-attract and activate the indicated cell types. Collectively, the cytokines and chemokinies up-regulated by PT-100 act to both increase effector cell numbers and concentrate the effector cells in the vicinity of the tumor.

TABLE 2
Function of cytokines and chemokines induced by PT-100

| | |
|---|---|
| Innate immunity | |
| Macrophage/monocyte | IL-1α/β*, IL-1R antagonist, MCP-2*, |
| | MARC/MCP-3*, MCP-5, JE* |
| Neutrophil | G-CSF*, MIP-2*, IL-8/KC*, ENA78*, LIX |
| NK & LAK | IL-1α/β*, IL-1R antagonist, IFN-β, Lymphotactin |
| Eosinophils | Eotaxin |
| T cell immunity | |
| T cells | IL-6*, IL-1α/β*, MARC/MCP-3, Lymphotactin |
| Activated T cells | MIG*, IP-10*, MDC |
| TIL | MIG* |
| Primed T helper cells | TARC |
| Anti-angiogenesis | |
| Endothelial cells | Thrombospondin, IP-10 |

*Produced in tumor mass

### Example 3

### Roles of Adaptive Immunity and Non-Adaptive (Innate) Immunity in PT-100 Activity Against WEHI 164 Tumors

Normal euthymic BALB/c mice (+/+) or athymic BALB/c mice that are congenitally deficient in mature T lymphocytes (nu/nu) were inoculated with WEHI 164 tumor cells. The mice were administered a 5 μg dose of PT-100 or saline (control) from day 2 until day 20 after tumor inoculation and tumor volumes (abscissae) were measured at the times indicated on the ordinate. Each treatment group contained 10 replicate mice. In both normal and athymic mice, PT-100 significantly inhibited tumor growth (p values shown were determined by Students t-test). In euthymic mice, however, tumors were completely rejected by day 20 in 40 per cent of the mice treated with PT-100 whereas tumor rejection was not observed in PT-100 treated athymic mice. In the WEHI 164 tumor model, tumor rejection never occurred spontaneously in control mice. The data indicate that PT-100 can stimulate non-adaptive immunity against the tumor, but specific T cell activity is required for tumor rejection. The data are consistent with a mechanism of action involving increased production of cytokines and chemokines in mice treated with PT-100 as described in Example 1.

### Example 4

### Effect of PT-100 and Rituxan in NOD/SCID Mouse Model of Burkitt's Non-Hodgkin's Lymphoma (NHL)

Immunodeficient NOD/SCID mice were inoculated with Namalwa cells derived from a Burkitt's NHL. The human lymphoma cells proliferated in the immunodeficient mice to form solid subcutaneous tumors. Mice were administered 1.5 mg of normal human lgG or 1.5 mg of a human CD20-specific antibody (Rituxan) on each of days 3, 5 and 7 after tumor inoculation. Additional treatments with 5 μg PT-100 administered twice daily from day 2 until day 20 after tumor inoculation were given as indicated in the Figure Legend. The four treatment groups each contained 4 or 5 replicate mice. The data represent mean tumor volumes (±SE). Control treatment with normal human IgG had no effect on tumor growth as compared with saline treatment (data not shown). Treatment with PT-100 and normal human IgG or with Rituxan alone, each significantly (p<0.05) inhibited tumor growth to a similar extent. Combined treatment with PT-100 and Rituxan inhibited tumor growth to a significantly (p<0.05) greater extent than did either treatment with PT-00 and normal human IgG or Rituxan alone. The data support the concept that when combined together, PT-100 and a tumor-specific antibody can have a greater growth inhibitory effect against a tumor than either treatment by itself.

### Example 5

### PT-00Increases IL-10 Gene Expression 30 Minutes After Oral Administration of PT-100 During Treatment Of WEHI 164 Tumors

Mice were inoculated with WEHI 164 cells, and starting 2 days later, administered (twice daily) a 5 μg dose of PT-100 or saline (control). RNA extracted from lymph nodes and tumors 30 minutes or 2 hours after the first dose of PT-100 on day 4 after tumor inoculation was processed, labeled and hybridized to Affymetrix GeneChips according to the manufacturer's instructions. The log ratios for expression values (PT-100 treated:saline treatment) have been plotted on the ordinates for the cytokine genes indicated on the abscissae. Zero values indicate that gene expression was either undetectable or unaffected by PT-100 treatment. The data compare induction of cytokine gene expression at 30 minutes and 2 hours after PT-100 administration in inguinal lymph nodes draining tumors.

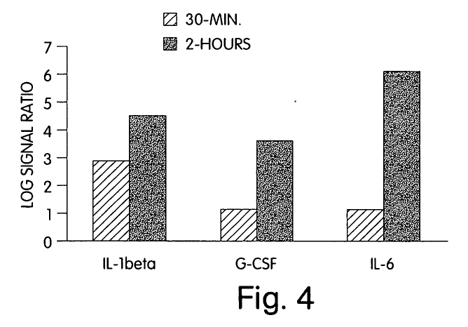

IL-1β mRNA levels increase in lymph nodes before those of other cytokines after oral PT-100 administration to mice, as shown in FIG. 4. This suggests that the timing of immunological challenge (e.g., vaccination or tumor-specific antibody infusion e.g., by direct and localized injection) relative to that of PT-100 may be important in some embodiments. For example, in some embodiments, PT-100 could be administered approximately 30 minutes earlier than administration of the antibody in order to ensure that IL-10 has been induced sufficiently prior to antibody administration.

### Example 6

### PT-100 Increases IL-1β Production by Splenic Tissue Without Affecting Serum Levels

BALB/c mice were orally administered 20 μg PT-100 or saline as indicated on the abscissae. Eight hours after PT-100 administration, IL-1β, G-CSF and KC levels were determined by ELISA (R&D Systems) of serum and extracts of spleens. Cytokine and chemokine levels are indicated on the ordinate as pg/ml or ng/ml of serum, and as pg/mg or ng/mg of protein in each spleen extract as determined by BCA protein assays (Pierce). The data indicate that a 20 [tg dose of PT-100 increases IL-1β levels in spleen tissue without increasing serum levels, whereas G-CSF and KC levels are increased in both spleen and serum.

Oral administration of PT-100 at doses within the range of 5- to 20-μg/mouse are sufficient to increase serum levels of certain growth factors, cytokines and chemokines and suppress tumor growth in BALB/c mice. The data demonstrate that oral administration of a 20-μg dose of PT-100 stimulates IL-1β protein production in the spleen without causing serum levels of IL-1 to increase.

Systemic administration of IL-1β by injection is associated with unacceptable toxicity. Therefore, if PT-100 treatment is to upregulate host defenses and function as an immunologically active adjuvant via IL-1β, it must be administered so as to avoid IL-1related side-effects. This can be achieved by using a dose of PT-100 that does not increase serum levels of IL-i1. In the above example, a 20 μg dose of PT-100 stimulated increased IL-1 production in the spleen, and increased serum levels of G-CSF and KC, but serum levels of IL-1 were unaltered. Accordingly, a dose of PT-100 determined to stimulate IL-1β production in lymphoid tissue without altering serum levels of IL-1β can be administered. Alternatively, a dose of PT-100 sufficient to increase serum levels of G-CSF and/or KC without increasing serum levels of IL-1β could also be administered. In human subjects, IL-8 is the homolog of KC.

### Example 7

### PT-100 Stimulated Increases in IL-1β G-CSF and KC are Dependent on IL-1β Signaling

Normal B6 mice (+/+) and congenic B6.129S7-Illr1^{tmllmx} mice with a targeted mutation of the IL-1 receptor-1 (−/−) were orally administered 40 or 160 μg of PT-100 or saline as indicated on the abscissae. 8-hours after PT-100 administration, IL-1β, G-CSF and KC levels were determined by ELISA of serum and extracts of spleen. Cytokine and chemokine levels are indicated on the ordinate as pg/ml or ng/ml of serum, and as pg/mg or ng/mg of protein in each spleen extract as determined by BCA protein assays. The data indicate that in the absence of the IL-1 receptor in mutant mice, PT-100 could still increase IL-1β levels in the spleen; but the magnitude of the response was greatly reduced compared to that in normal B6 mice. In the absence of the IL-1 receptor, the serum and splenic G-CSF and KC levels were essentially unaffected by PT-100 administration, indicating an absolute requirement for IL-1 signaling in order for PT-100 to stimulate production of these proteins.

The IL-1 receptor-1 is the only functional receptor for IL-1. Data indicate that in mice with a targeted mutation of the IL-1 receptor-1: PT-100 stimulated IL-1β production within splenic tissue was greatly reduced, and the G-CSF and KC responses to PT-100 were almost completely absent.

IL-1β can stimulate its own production via an autocrine loop. Therefore, the dependency of the splenic IL-1 response to PT-100 on the IL-1 receptor suggests that PT-100 acts rapidly in vivo to cause an increase in IL-1β in lymphoid tissue, and that this initial rise in IL-1β, itself provides the signal stimulating additional de novo IL-1β synthesis. Although not intending to be bound by any particular theory, it is possible that the cell types responsive to the compounds of Formula I co-express FAP, IL-1β and the IL-1 receptor-1.

Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

1. A method for stimulating an immune response in a subject comprising administering to a subject in need of immune stimulation an agent of Formula I, and an antibody or antibody fragment, in an amount effective to stimulate an immune response.

2. The method of claim 1, wherein the immune response is antibody dependent cell-mediated cytoxicity.

3. The method of claim 1, wherein the antibody or antibody fragment is an antibody.

4. The method of claim 1, wherein the antibody or antibody fragment is an anti-HER2 antibody.

5. The method of claim 4, wherein the anti-HER2 antibody is trastuzumab.

6. The method of claim 1, wherein the antibody or antibody fragment is an anti-CD20 antibody.

7. The method of claim 6, wherein the anti-CD20 antibody is rituximab.

8. The method of claim 1, wherein the antibody or antibody fragment is administered in a sub-therapeutic dose.

9. The method of claim 1, wherein the agent of Formula I is administered in a route of administration different from that of the antibody or antibody fragment.

10. The method of claim 1, wherein the agent of Formula I is administered orally and the antibody or antibody fragment is administered by injection.

11. The method of claim 1, wherein the agent of Formula I is administered prior to the antibody or antibody fragment.

12. The method of claim 11, wherein the agent of Formula I is administered 30 minutes to 8 hours prior to the antibody or antibody fragment.

13. The method of claim 11, wherein the agent of Formula I is administered 1 to 7 days prior to the antibody or antibody fragment.

14. The method of claim 1, wherein the agent of Formula I is administered substantially simultaneously with the antibody or antibody fragment.

15. The method of claim 1, wherein the agent of Formula I is administered after the antibody or antibody fragment.

16. The method of claim 15, wherein the agent of Formula I is administered 30 minutes to 8 hours after the antibody or antibody fragment.

17. The method of claim 15, wherein the agent of Formula I is administered 1 to 7 days after the antibody or antibody fragment.

18. A method for stimulating an immune response in a subject having or at risk of having cancer comprising administering to a subject in need of immune stimulation an agent of Formula I, and an antigen, in an amount effective to stimulate an antigen-specific immune response.

19-23. (canceled)

24. A method for stimulating an immune response in a subject comprising administering to a subject in need of immune stimulation an agent of Formula I, and an antigen, in an amount effective to stimulate an antigen-specific immune response, wherein the agent of Formula I is administered at a concentration of greater than 10⁻⁸M.

25. The method of claim 1, wherein the subject is a subject having or at risk of developing cancer.

26-29. (canceled)

30. The method of claim 1, wherein the subject is a subject having or at risk of developing an infectious disease.

31-80. (canceled)

81. A method of preventing an infectious disease in a subject at risk of developing an infectious disease comprising
identifying a subject at risk of developing an infectious disease, and
administering an agent of Formula I to the subject in an amount effective to induce IL-1.

82-89. (canceled)

90. A method for stimulating an immune response in a non-immunocompromised subject comprising
administering to a subject in need thereof an agent of Formula I, in an amount effective to induce IL-1.

91-96. (canceled)

97. A method for stimulating an immune response in an immunocompromised subject comprising administering to a subject in need thereof an agent of Formula I, in an amount effective to induce IL-1.

98-138. (canceled)

139. The method of claim 1, wherein the antibody or antibody fragment is administered on a first day of a seven day cycle and the agent of Formula I is administered twice a day on day two through day seven.

140-141. (canceled)

142. The method of claim 1, wherein the antibody or antibody fragment is conjugated to a toxin derived from plant, fungus, or bacteria.

143. (canceled)

144. The method of claim 1, wherein the antibody or antibody fragment is conjugated to a chemotherapeutic agent or a radioisotope.

145-165. (canceled)

166. The method of claim 1, wherein the antibody or antibody fragment is selected from the group consisting of Avastin (bevacizumab), BEC2 (mitumomab), Bexxar (tositumomab), Campath (alemtuzumab), CeaVac, Herceptin (trastuzumab), IMC-C225 (centuximab), LymphoCide (epratuzumab), MDX-210, Mylotarg (gemtuzumab ozogamicin), Panorex (edrecolomab), Rituxan (rituximab), Theragyn (pemtumomab), Zamyl, and Zevalin (ibritumomab tituxetan).

167. (canceled)

168. A method for treating a subject having or at risk of developing an IFN-responsive condition comprising
administering to a subject in need of such treatment an agent of Formula I in an amount effective to induce a therapeutically or prophylactically effective amount of IL-1 in the subject.

169-176. (canceled)

177. A method for treating a subject having or at risk of developing cancer comprising administering to a subject in need of such treatment an enzyme inhibitor selected from the group consisting of a tyrosine kinase inhibitor, a CDK inhibitor, a MAP kinase inhibitor, and an EGFR inhibitor, and an agent of Formula I in an amount effective to inhibit the cancer.

178-181. (canceled)

182. A method for treating a subject having or at risk of developing cardiovascular disease comprising
administering to a subject in need of such treatment an agent of Formula I in an amount effective to induce an effective amount of IL-1.

183. (canceled)

184. A method for preventing drug resistance in a subject having an infectious disease comprising
administering to a subject receiving an anti-microbial agent, an agent of Formula I in an amount effective to reduce the risk of resistance to the anti-microbial agent.

185-187. (canceled)

188. A method of shortening a vaccination course comprising
administering to a subject in need of immunization an agent of Formula I in an amount effective to induce an antigen-specific immune response to a vaccine administered in a vaccination course,
wherein the vaccination course is shortened by at least one immunization.

189-190. (canceled)

191. A method of shortening a vaccination course comprising
administering to a subject in need of immunization an agent of Formula I in an amount effective to induce an antigen-specific immune response to a vaccine administered in a vaccination course,
wherein the vaccination course is shortened by at least one day.

192-194. (canceled)

195. A method for stimulating an immune response in a subject having cancer comprising
administering to a subject in need of such treatment an agent of Formula I in an amount effective to stimulate an antigen-specific immune response, prior to and following a therapy selected from the group consisting of radiation, surgery and chemotherapy.

196. (canceled)

197. A method for stimulating an immune response in a subject at risk of developing cancer comprising
administering to a subject in need of such treatment an agent of Formula I in an amount effective to stimulate an antigen-specific immune response.

198-209. (canceled)

210. A method for modulating an immune response comprising
administering to a subject in need thereof an antibody or an antibody fragment on a first day of a seven day cycle, and administering to the subject an agent of Formula I on day 2 through to day 7 of the seven day cycle.

211-250. (canceled)

251. The method of claim 1, wherein the agent of Formula I is an agent of Formula II.

252. The method of claim 1, wherein the agent of Formula I is an agent of Formula III.

253. The method of claim 1, wherein the agent of Formula I is selected from the group consisting of L-Val-L-boroPro, L-Met-L-boroPro, and L-Ile-L-boroPro.

254. The method of claim 1, wherein the agent of Formula I is in a cyclic form.

255. The method of claim 1, wherein the agent of Formula I is administered in an amount that increases lymphoid tissue levels of IL-1, G-CSF or IL-8.

256. The method of claim 1, wherein the agent of Formula I is administered in an amount that does not increase serum IL-1 levels.

257. The method of claim 1, wherein the IL-1 is IL-1α or IL-1β.

258. The method of claim 1, wherein the subject is otherwise free of symptoms calling for hematopoietic stimulation.

259. The method of claim 1, wherein the agent of Formula I is administered on a routine schedule.

260. The method of claim 1, wherein the subject is HIV negative.

261. A composition comprising
an effective amount of an agent of Formula I and an antibody or antibody fragment.

262-289. (canceled)

290. A composition comprising
an effective amount of an agent of Formula I and a cancer antigen.

291-319. (canceled)

320. A composition comprising
an effective amount of an agent of Formula I and a microbial antigen,
wherein the agent of Formula I is formulated for administration at a dose of greater than 10⁻⁸M.

321-337. (canceled)

338. The method of claim 1, wherein the agent of Formula I is at least 96% pure L-isomer.

339. (canceled)
